SEQUENCE LISTING (68 sequences)

SEQ ID NO: 1 - protein, 237 aa, Artificial sequence
  Synthetic sequence
MAQVQLLESGGGLVQPGGSLTLACAASGFNFNNYNMNWVRQAPGRGLEWVAFISSAADTVHYADSVEGRFTISRDNAKNSLYLHMNSLRDEDTAVYYCARQLLYWGQGTLVTVSSGGGGGSQAVLTQPSSLSASPGASVSLTCTLRSGINVGAYRIYWYQQKPGSPPQFLLRYKSDSDKQQGSGVPSRFSGSRDASANAGILLISWLRSEDEADYYCAIWHSSAWVFGGGTQLTVLG

SEQ ID NO: 2 - protein, 115 aa, Artificial sequence
  Synthetic sequence
MAQVQLLESGGGLVQPGGSLTLACAASGFNFNNYNMNWVRQAPGRGLEWVAFISSAADTVHYADSVEGRFTISRDNAKNSLYLHMNSLRDEDTAVYYCARQLLYWGQGTLVTVSS

SEQ ID NO: 3 - protein, 116 aa, Artificial sequence
  Synthetic sequence
QAVLTQPSSLSASPGASVSLTCTLRSGINVGAYRIYWYQQKPGSPPQFLLRYKSDSDKQQGSGVPSRFSGSRDASANAGILLISWLRSEDEADYYCAIWHSSAWVFGGGTQLTVLG

SEQ ID NO: 4 - protein, 8 aa, Artificial sequence
  Synthetic sequence
GFNFNNYN

SEQ ID NO: 5 - protein, 8 aa, Artificial sequence
  Synthetic sequence
ISSAADTV

SEQ ID NO: 6 - protein, 6 aa, Artificial sequence
  Synthetic sequence
ARQLLY

SEQ ID NO: 7 - protein, 9 aa, Artificial sequence
  Synthetic sequence
SGINVGAYR

SEQ ID NO: 8 - protein, 7 aa, Artificial sequence
  Synthetic sequence
YKSDSDK

SEQ ID NO: 9 - protein, 9 aa, Artificial sequence
  Synthetic sequence
AIWHSSAWV

SEQ ID NO: 10 - protein, 232 aa, Artificial sequence
  Synthetic sequence
MAQVQLQESGGGLVQPGGSLRLSCAASGFTFSSYAMSWVRQAPGKGLEWVSAMSGSGGSTYYADSVKGRFTISRDNSKNTLYLQMNSLRAEDTAVYYCAKGVWFGELLPPFDYWGQGTLVTVSSGGGGSGGGGSGGGGSNFMLTQPHDPWPTAHSTQDTAHSTQDTAHSPQHTGHSTQHTGHSPQHTGHSTQDTAHSTQDTAHSTQPTAHSTQDTAHSPQHTGHGTQLTVLG

SEQ ID NO: 11 - protein, 124 aa, Artificial sequence
  Synthetic sequence
MAQVQLQESGGGLVQPGGSLRLSCAASGFTFSSYAMSWVRQAPGKGLEWVSAMSGSGGSTYYADSVKGRFTISRDNSKNTLYLQMNSLRAEDTAVYYCAKGVWFGELLPPFDYWGQGTLVTVSS

SEQ ID NO: 12 - protein, 93 aa, Artificial sequence
  Synthetic sequence
NFMLTQPHDPWPTAHSTQDTAHSTQDTAHSPQHTGHSTQHTGHSPQHTGHSTQDTAHSTQDTAHSTQPTAHSTQDTAHSPQHTGHGTQLTVLG

SEQ ID NO: 13 - protein, 8 aa, Artificial sequence
  Synthetic sequence
GFTFSSYA

SEQ ID NO: 14 - protein, 8 aa, Artificial sequence
  Synthetic sequence
MSGSGGST

SEQ ID NO: 15 - protein, 15 aa, Artificial sequence
  Synthetic sequence
AKGVWFGELLPPFDY

SEQ ID NO: 16 - protein, 6 aa, Artificial sequence
  Synthetic sequence
GGGGGS

SEQ ID NO: 17 - protein, 15 aa, Artificial sequence
  Synthetic sequence
GGGGSGGGGSGGGGS

SEQ ID NO: 18-23 - skipped (6 entries)

SEQ ID NO: 24 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 25 - protein, 11 aa, Homo sapiens
EEAENTLQSFR

SEQ ID NO: 26 - protein, 10 aa, Homo sapiens
QDVDNASLAR

SEQ ID NO: 27 - DNA, 20 bp, Artificial sequence
  Synthetic sequence
aggacctgctcaatgtcaag

SEQ ID NO: 28 - DNA, 21 bp, Artificial sequence
  Synthetic sequence
tcttccctgaaaaccttgtcc

SEQ ID NO: 29 - DNA, 20 bp, Artificial sequence
  Synthetic sequence
gtacaaccgcttcctcttcc

SEQ ID NO: 30 - DNA, 19 bp, Artificial sequence
  Synthetic sequence
tggggccttcaccatggat

SEQ ID NO: 31 - DNA, 18 bp, Artificial sequence
  Synthetic sequence
gcaaccccttcctcagtc

SEQ ID NO: 32 - DNA, 21 bp, Artificial sequence
  Synthetic sequence
cacggatggagaaaagtttgg

SEQ ID NO: 33 - DNA, 22 bp, Artificial sequence
  Synthetic sequence
catctctgtactgaaccccaac

SEQ ID NO: 34 - DNA, 21 bp, Artificial sequence
  Synthetic sequence
ccctcagcaaatatcactccg

SEQ ID NO: 35 - DNA, 22 bp, Artificial sequence
  Synthetic sequence
cttcctggatgggatagtggac

SEQ ID NO: 36 - DNA, 21 bp, Artificial sequence
  Synthetic sequence
gtgctgggctatcgttatgtt

SEQ ID NO: 37 - DNA, 22 bp, Artificial sequence
  Synthetic sequence
tcttgtctcaatcacccttcag

SEQ ID NO: 38 - DNA, 22 bp, Artificial sequence
  Synthetic sequence
ggccctgtaattggaatgagtc

SEQ ID NO: 39 - DNA, 22 bp, Artificial sequence
  Synthetic sequence
aagaatggaagagtcagtgtgg

SEQ ID NO: 40 - DNA, 28 bp, Artificial sequence
  Synthetic sequence
tgaggcgctgtcgtcatcgatttctccc

SEQ ID NO: 41 - DNA, 21 bp, Artificial sequence
  Synthetic sequence
atggatgttgatggctactgg

SEQ ID NO: 42 - DNA, 19 bp, Artificial sequence
  Synthetic sequence
cgaaacgcttcacttccaa

SEQ ID NO: 43 - DNA, 26 bp, Artificial sequence
  Synthetic sequence
cacgtcgtagcaaaccaccaagtgga

SEQ ID NO: 44 - DNA, 20 bp, Artificial sequence
  Synthetic sequence
acggaccccaaaagatgaag

SEQ ID NO: 45 - DNA, 20 bp, Artificial sequence
  Synthetic sequence
gtcttcctgggcaagcagta

SEQ ID NO: 46 - DNA, 20 bp, Artificial sequence
  Synthetic sequence
gtaacccgttgaaccccatt

SEQ ID NO: 47 - DNA, 20 bp, Artificial sequence
  Synthetic sequence
aggctggtttctcgaatctg

SEQ ID NO: 48 - DNA, 21 bp, Artificial sequence
  Synthetic sequence
agtcacagtctcgttcaacag

SEQ ID NO: 49 - DNA, 21 bp, Artificial sequence
  Synthetic sequence
ttgatcttggctgtctccttc

SEQ ID NO: 50 - DNA, 22 bp, Artificial sequence
  Synthetic sequence
gccaatggcaaagatgacggag

SEQ ID NO: 51 - DNA, 21 bp, Artificial sequence
  Synthetic sequence
acccgtttctcaccaatatcg

SEQ ID NO: 52 - DNA, 22 bp, Artificial sequence
  Synthetic sequence
tggatgcgatggtattaagctc

SEQ ID NO: 53 - DNA, 22 bp, Artificial sequence
  Synthetic sequence
ccatgtagctcaggtagacaac

SEQ ID NO: 54 - DNA, 22 bp, Artificial sequence
  Synthetic sequence
cacccgatcttcatcttatccc

SEQ ID NO: 55 - DNA, 21 bp, Artificial sequence
  Synthetic sequence
gcacagacgatgaacatcagc

SEQ ID NO: 56 - DNA, 20 bp, Artificial sequence
  Synthetic sequence
gaatcgggtacggagcttgg

SEQ ID NO: 57 - DNA, 21 bp, Artificial sequence
  Synthetic sequence
ctaaacctcgtaatgctcccc

SEQ ID NO: 58 - DNA, 21 bp, Artificial sequence
  Synthetic sequence
ccaagatccaactacgagctt

SEQ ID NO: 59 - DNA, 21 bp, Artificial sequence
  Synthetic sequence
gggagtgttgatgtcagtgtg

SEQ ID NO: 60 - DNA, 20 bp, Artificial sequence
  Synthetic sequence
acctgctccactgccttgct

SEQ ID NO: 61 - DNA, 20 bp, Artificial sequence
  Synthetic sequence
ttctgactctggacacttgc

SEQ ID NO: 62 - DNA, 21 bp, Artificial sequence
  Synthetic sequence
tgagcctatattgctgtggct

SEQ ID NO: 63 - DNA, 23 bp, Artificial sequence
  Synthetic sequence
tgggagtagacaaggtacaaccc

SEQ ID NO: 64 - DNA, 20 bp, Artificial sequence
  Synthetic sequence
ttctccacagccacaatgag

SEQ ID NO: 65 - DNA, 20 bp, Artificial sequence
  Synthetic sequence
ctggacagaaaccccacttc

SEQ ID NO: 66 - DNA, 20 bp, Artificial sequence
  Synthetic sequence
ccatccaatcggtagtagcg

SEQ ID NO: 67 - DNA, 711 bp, Artificial sequence
  Synthetic sequence
atggcacaggtgcagctgttggagtctgggggaggtttggtgcagccgggggggtccctgacactcgcctgtgcagcctctggattcaacttcaacaactataacatgaactgggtccgccaggctccagggaggggactggagtgggttgccttcattagtagtgctgctgataccgtgcactacgcagactctgtggagggccgattcaccatctccagagacaacgccaagaactcactgtatctacatatgaacagcctgagagacgaagacacggctgtttattactgcgcgaggcaattactctactggggccagggcaccctggtcaccgtctcctcaggcggcggtggcggatcccaggctgtgctcactcagccgtcttccctctctgcatctcctggagcatcagtcagtctcacctgcactttacgcagtggcatcaatgttggtgcctacaggatatactggtaccagcagaagccagggagtcctccccagttcctcctgaggtacaaatcagactcagataagcagcagggctctggagtccccagccgcttctctggatccagagatgcttcggccaatgcagggattttactcatctcttggctccggtctgaggatgaggctgactattactgtgcgatttggcacagcagcgcttgggtgttcggcggagggacccagctcaccgttttaggt

SEQ ID NO: 68 - DNA, 696 bp, Artificial sequence
  Synthetic sequence
atggcacaggtgcagctgcaggagtccgggggaggcttggtacagcctggggggtccctgagactctcctgtgcagcctctggattcacctttagcagctatgccatgagctgggtccgccaggctccagggaaggggctggagtgggtctcagctatgagtggtagtggtggtagtacatactacgcagactccgtgaagggccggttcaccatctccagagacaattccaagaacacgctgtatctgcaaatgaacagcctgagagctgaggacacggctgtgtattactgtgcgaaaggggtatggttcggggagttattaccaccctttgactactggggccagggaaccctggtcaccgtctcctcaggcggcggcggctctggcggaggtggcagcggcggtggcggatccaattttatgctgactcagccccatgacccatggcccacagcccacagcacacaggacacagcacacagcacacaggacacagcccacagcccacagcacacaggacacagcacacagcacacaggacacagcccacagcacacaggacacagcacacaggacacagcccacagcacacaggacacagcccacagcacacagcccacagcccacagcacacaggacacagcccacagcccacagcacacaggacacgggacccagctcaccgttttaggt

(19) United States
(12) Patent Application Publication
(10) Pub. No.: US 2020/0140851 A1
(43) Pub. Date: May 7, 2020

(54) Methods and Compositions Related to In Vivo Selection of Functional Molecules

(71) Applicant: The Scripps Research Institute, La Jolla, CA (US)

(72) Inventors: Kyung Ho Han, San Diego, CA (US); Richard A. Lerner, La Jolla, CA (US)

(21) Appl. No.: 16/282,705

(22) PCT Filed: Aug. 18, 2017
(86) PCT No.: PCT/US2017/047585

§ 371 Date: Feb. 22, 2019

Related U.S. Application Data
(60) Provisional application No. 62/378,350, filed on Aug. 23, 2016.

(51) Int. Cl. C12N 15/10; G01N 33/50; C07K 16/18

(52) U.S. Cl. CPC C12N 15/1037; G01N 33/5044; C07K 2317/70; C07K 2317/565; C07K 2317/14; C07K 16/18

(57) ABSTRACT

The Invention provides in vivo selection methods for identifying modulating agents (e.g., antibodies or polypeptides) that promote cellular differentiation and migration. The methods utilize a combinatorial agent library (e.g., antibodies expressed via lentiviral vectors) that are expressed in a population of to-be modulated cells (e.g., stem cells), which are then introduced into, the body of a non-hum an animal (e.g., mouse). This is followed by examining an organ or tissue of interest (e.g., brain) of the manipulated animal for the presence of a modulating agent and/or a specific phenotype. The; invention also provides specific antibody agent, that can induce differentiation of stem cells into microglia and migration into the brain. Further provided in the invention are therapeutic applications of the microglia-inducing antibodies.

### CROSS-REFERENCE TO RELATED APPLICATIONS

The subject patent application claims the benefit of priority to U.S. Provisional Patent Application No. 62/378,350 (filed Aug. 23, 2016). The full disclosure of the priority application is incorporated herein by reference in its entirety and for all purposes.

### BACKGROUND OF THE INVENTION

Cell migration is central to the embryonic development and maintenance of all organisms. In spite of its immense importance and much study, our knowledge of this process is still incomplete. For instance, in the adult we still do not have a comprehensive understanding of which cells can migrate, where they go, and what they differentiate into when they reach their destination. In clinical settings, one must induce cells of a desired phenotype that also properly integrate into the tissue of interest in order to repair damaged tissues in the adult. This is undoubtedly a formidable two-step event. Currently, there is no effective means for reconstituting damaged organ systems in the adult.

There is a need in the art for effective means for recapitulating embryonic events for clinical applications. The present invention is directed to this and other needs.

### SUMMARY OF THE INVENTION

In one aspect, the invention provides methods for identifying a functional modulating agent (e.g., antibody) that can induce stem cells to differentiate and migrate to a specific organ or tissue. Some of these methods involve (a) expressing in a population of stem cells a library of candidate antibodies or antigen-binding fragments thereof to produce a heterogeneous population of modified, antibody-expressing hematopoietic stem cells, (b) introducing the heterogeneous population of antibody-expressing hematopoietic stem cells into a non-human animal, and (c) detecting in a specific organ or tissue of the non-human animal the presence of a sequence encoding a candidate antibody. These procedures allow one to identify functional modulating agents (e.g., antibodies) that can induce stem cell differentiation and migration to a specific organ or tissue.

In some of these methods, the employed stem cells are bone marrow cells. Some of these methods employ a non-human animal that is a mouse. In some of these methods, the non-human animal is lethally irradiated prior to introducing antibody-expressing hematopoietic stem cells into the animal. In some methods, the antibody-expressing hematopoietic stem cells are introduced into the animal via injection. In some methods, the specific tissue targeted is a tissue from brain, heart, liver or spleen. In some methods, the employed library of candidate antibodies is a combinatorial library of scFv or scFv-Fc molecules. In some of these methods, the combinatorial antibody library is expressed in the stem cells via a lentiviral vector or a retroviral vector. Some methods of the invention additionally involve determining amino acid sequences of heavy chain and light chain variable regions of the identified candidate antibody.

In another aspect, the invention provides antibodies or antigen-binding fragments that have the same binding specificity as that of a second antibody. The second antibody contains (a) heavy chain CDRs 1-3 and light chain CDRs 1-3 sequences that are respectively identical to GFN FNNYN (SEQ ID NO:4), ISSAADTV (SEQ ID NO:5), ARQLLY (SEQ ID NO:6), SGIN VGAYR (SEQ ID NO:7), YKSDSDK (SEQ ID NO:8), and AIWHSSAWV (SEQ ID NO:9), (b) heavy chain and light chain variable region sequences respectively shown in SEQ ID NOs:2 and 3, or (c) heavy chain and light chain variable region sequences respectively shown in SEQ ID NOs:11 and 12. Some of these antibodies or antigen-binding fragments contain heavy chain CDRs 1-3 sequences that are substantially identical, respectively, to (a) GFN FNNYN (SEQ ID NO:4), ISSAADTV (SEQ ID NO:5), and ARQLLY (SEQ ID NO:6) or (b) GFTFSSYA (SEQ ID NO:13), MSGSGGST (SEQ ID NO: 14), and AKGVWFGELLPPFDY (SEQ ID NO: 15). Some of these antibodies or antigen-binding fragments further contain light chain CDRs 1-3 sequences that are substantially identical, respectively, to SGIN VGAYR (SEQ ID NO:7), YKSDSDK (SEQ ID NO:8), and AIWHSSAWV (SEQ ID NO:9). Some of the antibodies or antigen-binding fragments contain a heavy chain CDR sequence selected from the group consisting of SEQ ID NOs:4-6 and 13-15. Some of these molecules additionally contain a light chain CDR sequence selected from the group consisting of SEQ ID NOs:7-9.

Some antibodies or antigen-binding fragments of the invention contain heavy chain CDRs 1-3 sequences that are respectively identical to (1) SEQ ID NOs:4-6 or (2) SEQ ID NOs:13-15. Some of these molecules contain heavy chain CDRs 1-3 and light chain CDRs 1-3 sequences respectively shown in SEQ ID NOs:4-6 and SEQ ID NOs:7-9. Some antibodies or antigen-binding fragments of the invention contain a light chain CDR sequence selected from the group consisting of SEQ ID NOs:7-9. Some of these molecules additionally contain a heavy chain CDR sequence selected from the group consisting of SEQ ID NOs:4-6 and 13-15. Some of these molecules contain light chain CDRs 1-3 sequences that are respectively identical to SEQ ID NOs:7-9. Some antibodies or antigen-binding fragments of the invention contain a heavy chain variable region sequence that is at least 90% identical to SEQ ID NO:2 or 11. Some antibodies or antigen-binding fragments of the invention contain a light chain variable region sequence that is at least 90% identical to SEQ ID NO:3 or 12. Some antibodies or antigen-binding fragments of the invention contain a heavy chain variable region sequence and a light chain variable region sequence that are at least 90% identical, respectively, to (1) SEQ ID NOs:2 and 3 or (2) SEQ ID NOs: 11 and 12. Some antibodies or antigen-binding fragments of the invention contain a heavy chain variable region sequence and a light chain variable region sequence, one or both of which are identical to a heavy chain variable region sequence and a light chain variable region sequence respectively shown in (1) SEQ ID NOs:2 and 3 or (2) SEQ ID NOs:11 and 12. Some of these molecules contain a heavy chain variable region sequence and a light chain variable region sequence respectively shown in (1) SEQ ID NOs:2 and 3 or (2) SEQ ID NOs:11 and 12.

Some antibodies or antigen-binding fragments of the invention are scFv fragments that contain heavy chain and light chain variable region sequences connected via a linker sequence. In some of these molecules, the linker sequence contains GGGGGS (SEQ ID NO: 16) or GGGGSGGGGSGGGGS (SEQ ID NO:17). Some of the molecules contain an amino acid sequence shown in SEQ ID NO:1 or 10. In various embodiments, the antibodies or antigen-binding fragments of the invention are IgA1, IgA2, IgD, IgE, IgG1, IgG2, IgG3, IgG4, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, non-depleting IgG, diabodies, or bivalent antibodies. For example, the molecule can be an IgG selected from the group consisting of IgG1, IgG2, IgG3, IgG4, and synthetic IgG. In some other embodiments, the molecule is a Fab, a scFv, or a dsFv. Some of the scFv fragments of the invention are further conjugated to an Fc domain or a label moiety.

In a related aspect, the invention provides methods for treating a brain disorder or injury in a subject. These methods entail administering to a subject afflicted with or at risk of developing the brain disorder or injury a pharmaceutical composition comprising a therapeutically effective amount of a microglia-inducing antibody described herein or a stem cell population (e.g., bone marrow cells) that is first treated with the antibody. In some embodiments, the subject to be treated has or is at risk of developing dementia, esp. Alzheimer's disease. In some of these embodiments, the employed stem cell population is isolated from the very subject in need of treatment.

A further understanding of the nature and advantages of the present invention may be realized by reference to the remaining portions of the specification and claims.

### DESCRIPTION OF THE DRAWINGS

FIG. 1 shows in vivo selection of an antibody that induces differentiation and migration of mouse and human HSCs. Scheme of the phenotype selection in vivo. Genes from a human scFv phage library (10⁸ members) were cloned into a lentiviral vector to make a lentiviral intra-body library in which antibody molecules are attached to the plasma membrane and displayed on the cell surface. Total mouse bone marrow cells were infected with the antibody library in vitro, and transplanted into lethally irradiated C57BL/6J mice. The system is autocrine based because each cell has a different antibody and the putative target. After 10 days, the mouse brains were harvested and analyzed by PCR to identify any antibody genes from cells that traffic to the brain.

FIG. 2 shows that an agonist antibody regulates cell migration. Shown in the figure is a scheme of the in vivo migration of cells from the bone marrow to the brain. A pool of the antibody genes from all cells that had migrated to the brain migrated library and a single antibody gene (B1 Ab) that was the most abundant from the pool were separately reinserted into lentiviral vectors and used to infect total mCherry⁺ mouse bone marrow cells.

FIG. 3 shows 3D image of network formed by migrating microglial cells. The microglia cells were stained with DAPI, mCherry and TMEMI19 antibody. The confocal 3D images were analyzed by using by IMARS software. Scale bars=10, 20 and 50 μm.

FIG. 4 shows antibody induced differentiation of HSCs into microglia. (A) Scheme of the hematopoietic stem cell differentiation by B1 Ab. In the presence of purified B1 antibody for 2 weeks, total mouse bone marrow and human CD34⁺ cells differentiated into cells with the morphology of microglia. H indicates human CD34 cells. M indicates mouse bone marrow. (B) Human CD34⁺ cells treated with B1 or isotype control antibody were harvested after 2 weeks to extract total RNA for qRT-PCR analysis. A distinct microglia mRNA expression profile was revealed when relative mRNA levels of oligodendrocyte (Olig1, Olig2, and MOG), astrocyte (GFAP, SLCA2, and ALDHILA), and microglia (CX3CR1, IBA1, CD11 lb, CD68, F4/80, TMEM119, GPR84, and HEXB) gene markers were compared by qRT-PCR. (C) In addition, the B1 antibody-differentiated human CD34⁺ cells expressed the microglia surface proteins TMEM119, CD11b, and CX3CR1, as determined by immunofluorescence cytochemistry with antibodies to these markers. Nuclei were stained with DAPI.

FIG. 5 shows identification of a novel antigen recognized by the B1 antibody. (A) Cell lysates of human CD34⁺ cells were incubated with the B1 Ab to immune-precipitate binding antigens. Immune-precipitated elutes were separated by SDS/PAGE and subjected to mass-spectrometry (MS) analysis. Nano-LC-MS/MS analysis identified three candidate hits as potential target antigens. One of the antigens is human vimentin (SEQ ID NO:24). The VIM peptides identified by the MS study are residues 197-207, EEAENTLQSFR (SEQ ID NO:25), and residues 208-217 QDVDNASLAR (SEQ ID NO:26). (B) The B1 Ab bound to commercial VIM protein and C57BL/6J mouse bone marrow lysates in Western blots. Importantly, no protein band was observed when the B1 Ab was blotted with bone marrow lysates from VIM-deficient mice (JAX stock 025692). (C) Surface expression of VIM on human CD34⁺ cells was confirmed by confocal microscopy using DAPI, B1 or commercial VIM antibodies, or antibody against CD34. (D, E) Human CD34⁺ cells were treated with B1 Ab or control isotype antibody, and cell lysates were assessed by Western blotting using antibodies against nonphosphorylated and phosphorylated (p-) AKT, ERK, p38 and VIM S38.

FIG. 6 shows that B Ab induced M2-polarization of differentiated microglia. (A) Mouse bone marrow cells were incubated with B1 Ab for 2 weeks. Cells were then harvested to extract total RNA for qRT-PCR analysis. iNOS, TNFα, and IL1β were used as M1 gene markers, and ARG1, IL10, and CD206 were used for M2 markers. qRT-PCR suggested M2 polarization of the differentiated microglia with higher mRNA expression of M2 gene markers. (B) For flow cytometric analysis of the microglia resulting from the cells that were differentiated by the B1 Ab induced to differentiate, the cells were first identified as CD45^{low-int}CD11b⁺TMEM119⁺CX3CR1⁺ and then gated further to find M1/M2 subpopulations. M1-specific antibodies against MHCII and CD86, and M2-specific antibodies against CD14, CD36, and CD206 were used to assess M1/M2 polarization. The cells treated with antibody expressed surface markers consistent with M2-polarized microglia. (C) A functional phagocytosis assay was performed on the microglia-like cells that differentiated from human CD34⁺ cells. To assay function, we determined if the cells were capable of engulfing fluorescently labeled beads. After 85 minutes, the microglia had engulfed the beads.

FIG. 7 shows phylogenetic tree created by DNA sequencing analysis of antibodies in cells that had migrated in different organs. Antibody genes were recovered from tissues using PCR. A total of 60 antibody genes were isolated from the brain, heart, liver, and spleen and sequenced. 20 brain genes could be grouped into 4 major homologs. The B1 gene was most abundant as it appeared 6 times in the brain, but not in any other tissue.

FIG. 8 displays some of the real time PCR primer sequences described herein. Shown in the figure are forward (SEQ ID NOs:27-46) and reverse primers (SQE ID NOs:47-66) used for 20 genes.

FIG. 9 shows that bone marrow cell expressing B1 Ab protect APP/PS1 mice from neurodegeneration. (A) Plaque deposition showing a protective effect of B1 Ab treatment relative to control. Total mouse bone marrow cells were infected with lentivirus encoded B1 Ab or untreated cells (control) and transplanted into lethally irradiated 8 weeks old APP/PS1 mice (7/group). Significant differences between B1Ab treated and control mice are indicated by ** p<0.005 (Student's t-test). (B) Total wild-type C57BL/6J mouse bone marrow cells were infected with lentivirus encoded B1 Ab or untreated cells (control) and transplanted into lethally irradiated 8 weeks old APP/PS1 mice. After 2 weeks (2 month old) and 5 months (6 month old) post transfer, the mice were perfused with PBS, and brains were harvested and fixed in 2% PFA. Brain sections (50 rpm) were stained with IBA1 for microglia and GFAP for astrocytes and analyzed by confocal microscopy. Yellow fluorescent units (top panel) of IBA1 signal from brain coronal sections obtained by confocal microscopy were quantified using imagePro software. Staining of the hippocampus with GFAP (bottom) is shown. Scale bar=1 mm. Significant differences between B1Ab treated and control mice are indicated by * p<0.05 (Student's t-test).

### DETAILED DESCRIPTION OF THE INVENTION

### I. Overview

The invention is predicated in part on the development by the present inventors of migration-based selection methods which enable identification from cells expressing a combinatorial library of antibodies rare cells that have the appropriate phenotype as well as the ability to migrate and properly integrate into target tissues. Unlike known selection formats where one must separate induced from un-induced cells, the migration-based selection methods of the invention have as the end point the cell population of interest being detected in a different location. Rather than isolating phenotypically interesting cells from a mixture, the selection format of the invention enables one to study cell populations that, for physiological reasons, are enriched by self-separation. As exemplification, the inventors employed this method combined with adoptive transfer protocols to isolate antibodies that induce hematopoietic stem cells (HSCs) from bone marrow cells to differentiate and then selectively migrate to different tissue compartments. By adding control of migration to the ability of intracellular antibodies to induce differentiation of cells, the methods of the invention can facilitate reconstitution of organ systems in vivo.

As exemplification, the methods of the invention allow the inventors to select modulating agents such as antibody agonists that induce bone marrow stem cells to differentiate into microglia. It was observed that the induced cells have the morphology of microglia, are strongly phagocytic, and contain multiple markers associated with microglia. Importantly, the induced cells migrated to the brain where they form networks typical of microglia. Specific antibodies that induced bone marrow cells to differentiate into microglia and migrate into the brain (e.g., antibody B1) are exemplified herein. Also, exogenous soluble antibody added to stem cells elicits the same differentiation program. Specifically, the in vitro induced microglial have anti-inflammatory phenotype and are phagocytic as expected. The inventors additionally discovered that one identified antibody (antibod) B1) specifically recognizes vimentin (VIM), an intermediate filament protein known to be involved in polar morphology. Furthermore, it was observed that microglia-like cells induced by the specific antibodies disclosed herein were capable of migrating to the brain in the absence of irradiation, and that the induced microglia-like cells were also able to reduce Aβ plaques in APP/PS1 mouse model for the Alzheimer's disease. Thus, by promoting microglia differentiation and migration, the antibodies and cell populations described herein can have therapeutic applications in treating tissue damages associated with brain injuries or infections.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.). Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” or “antigen-binding fragment” refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate, camelid, avian or pisces species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region is comprised of three domains, C_{H1}, C_{H2} and C_{H3}. Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (C1q) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

Antibodies to be used in the invention also include antibody fragments or antigen-binding fragments which contain the antigen-binding portions of an intact antibody that retain capacity to bind the cognate antigen. Examples of such antibody fragments include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} domain (see, e.g., Ward et al., Nature 341:544-546, 1989); and (vii) an isolated complementarity determining region (CDR).

Antibodies suitable for practicing the present invention also encompass single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

Antibodies that can be used in the practice of the present invention also encompass single domain antigen-binding units which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The various antibodies or antigen-binding fragments described herein can be produced by enzymatic or chemical modification of the intact antibodies, or synthesized de novo using recombinant DNA methodologies, or identified using phage display libraries. Methods for generating these antibodies or antigen-binding molecules are all well known in the art. For example, single chain antibodies can be identified using phage display libraries or ribosome display libraries, gene shuffled libraries (see, e.g., McCafferty ct al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plickthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nature Struct. Biol. 11:500-515, 2002; Ghahroudi et al., FEBS Letters 414:521-526, 1997; and Bond et al., J Mol Biol. 332:643-55, 2003. Other types of antigen-binding fragments (e.g., Fab, F(ab′)₂ or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, N.Y., 1998.

An intrabody is an antibody that works within the cell to bind to an intracellular protein. Due to the lack of a reliable mechanism for bringing antibodies into the cell from the extracellular environment, this typically requires the expression of the antibody within the target cell. Because antibodies ordinarily are designed to be secreted from the cell, intrabodies require special alterations, including the use of single-chain antibodies (scFvs), modification of immunoglobulin V_{L} domains for hyperstability, selection of antibodies resistant to the more reducing intracellular environment, or expression as a fusion protein with maltose binding protein or other stable intracellular proteins.

Binding affinity is generally expressed in terms of equilibrium association or dissociation constants (Kₐ or K_{d}, respectively), which are in turn reciprocal ratios of dissociation and association rate constants (k_{d} and kₐ, respectively). Thus, equivalent affinities may correspond to different rate constants, so long as the ratio of the rate constants remains the same.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences (e.g., antibody chains), “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing two polypeptides or mixing a population of antibodies with a population of cells in a test tube or growth medium. Contacting can also occur in a cell or in situ, e.g., contacting two polypeptides in a cell by coexpression in the cell of recombinant polynucleotides encoding the two polypeptides, or in a cell lysate. Contacting may also occur in vivo inside a human body or the body of a non-human animal.

A “fusion” protein or polypeptide refers to a polypeptide comprised of at least two polypeptides and a linking sequence or a linkage to operatively link the two polypeptides into one continuous polypeptide. The two polypeptides linked in a fusion polypeptide are typically derived from two independent sources, and therefore a fusion polypeptide comprises two linked polypeptides not normally found linked in nature.

“Heterologous”, when used with reference to two polypeptides, indicates that the two are not found in the same cell or microorganism in nature. Allelic variations or naturally-occurring mutational events do not give rise to a heterologous biomolecule or sequence as defined herein. A “heterologous” region of a vector construct is an identifiable segment of polynucleotide within a larger polynucleotide molecule that is not found in association with the larger molecule in nature. Thus, when the heterologous region encodes a mammalian gene, the gene will usually be flanked by polynucleotide that does not flank the mammalian genomic polynucleotide in the genome of the source organism.

A “ligand” is a molecule that is recognized by a particular antigen, receptor or target molecule. Examples of ligands that can be employed in the practice of the present invention include, but are not restricted to, agonists and antagonists for cell membrane receptors, toxins and venoms, viral epitopes, hormones, hormone receptors, polypeptides, peptides, enzymes, enzyme substrates, cofactors, drugs (e.g. opiates, steroids, etc.), lectins, sugars, polynucleotides, nucleic acids, oligosaccharides, proteins, and monoclonal antibodies.

“Linkage” refers to means of operably or functionally connecting two biomolecules (e.g., polypeptides or polynucleotides encoding two polypeptides), including, without limitation, recombinant fusion, covalent bonding, disulfide bonding, ionic bonding, hydrogen bonding, and electrostatic bonding. “Fused” refers to linkage by covalent bonding. A “linker” or “spacer” refers to a molecule or group of molecules that connects two biomolecules, and serves to place the two molecules in a preferred configuration with minimal steric hindrance.

Microglial cells (microglia) are a type of glial cells located throughout the brain and spinal cord. Microglia account for 10-15% of all cells found within the brain. As the resident macrophage cells, they act as the first and main form of active immune defense in the central nervous system (CNS). Microglia (and other glia including astrocytes) are distributed in large non-overlapping regions throughout the CNS. Microglia are key cells in overall brain maintenance—they are constantly scavenging the CNS for plaques, damaged or unnecessary neurons and synapses, and infectious agents. Since these processes must be efficient to prevent potentially fatal damage, microglia are extremely sensitive to even small pathological changes in the CNS. This sensitivity is achieved in part by the presence of unique potassium channels that respond to even small changes in extracellular potassium.

Multiplicity of infection or MOI refers to the ratio of infectious agents (e.g. phage or virus) to infection targets (e.g., cell). For example, when referring to a group of cells inoculated with infectious virus particles, the multiplicity of infection or MOI is the ratio of the number of infectious virus particles to the number of target cells present in a defined space.

The term “operably linked” when referring to a nucleic acid, refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide of the embodiments of the invention may be polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as “nucleotide polymers.

Polypeptides are polymer chains comprised of amino acid residue monomers which are joined together through amide bonds (peptide bonds). The amino acids may be the L-optical isomer or the D-optical isomer. In general, polypeptides refer to long polymers of amino acid residues, e.g., those consisting of at least more than 10, 20, 50, 100, 200, 500, or more amino acid residue monomers. However, unless otherwise noted, the term polypeptide as used herein also encompass short peptides which typically contain two or more amino acid monomers, but usually not more than 10, 15, or 20 amino acid monomers.

Proteins are long polymers of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies. In some embodiments, the terms polypeptide and protein may be used interchangeably.

Unless otherwise noted, the term “receptor” broadly refers to a molecule that has an affinity for a given ligand. Receptors may-be naturally-occurring or manmade molecules. Also, they can be employed in their unaltered state or as aggregates with other species. Receptors may be attached, covalently or noncovalently, to a binding member, either directly or via a specific binding substance. A typical example of receptors which can be employed in the practice of the invention is cell surface signaling receptor.

Stem cell are cells that have the ability to divide and create an identical copy of themselves, a process called self-renewal, and can also divide to form cells that mature into cells that make up every type of tissue and organ in the body. Pluripotent stem cells are cells that have the potential of taking on many fates in the body, including all of the more than 200 different cell types. Embryonic stem cells are pluripotent, as are induced pluripotent stem (iPS) cells that are reprogrammed from adult tissues.

Embryonic stem cells are derived from pluripotent cells, which exist only at the earliest stages of embryonic development. In humans, these cells no longer exist after about five days of development. When isolated from the embryo and grown in a lab dish, pluripotent cells can continue dividing indefinitely. These cells are known as embryonic stem cells.

Adult stem cells are found in the various tissues and organs of the human body. They are thought to exist in most tissues and organs where they are the source of new cells throughout the life of the organism, replacing cells lost to natural turnover or to damage or disease. Adult stem cells are committed to becoming a cell from their tissue of origin, and can't form other cell types. They are therefore also called tissue-specific stem cells. They have the broad ability to become many of the cell types present in the organ they reside in. For example, adult blood-forming stem cells in the bone marrow can give rise to any of the red or white cells of the blood system, and adult stem cells in the intestine can form all the cell types of the intestinal lining.

Hematopoietic stem cells (HSCs) are cells isolated from the blood or bone marrow that can renew itself, can differentiate to a variety of specialized cells, can mobilize out of the bone marrow into circulating blood, and can undergo programmed cell death, called apoptosis—a process by which cells that are detrimental or unneeded self-destruct.

Induced pluripotent stem cell, or iPS cells, are cells taken from any tissue (usually skin or blood) from a child or adult and is genetically modified to behave like an embryonic stem cell. As the name implies, these cells are pluripotent, which means that they have the ability to form all adult cell types.

The term “subject” refers to human and non-human animals (especially non-human mammals). In addition to human, it also encompasses other non-human animals such as cows, horses, sheep, pigs, cats, dogs, mice, rats, rabbits, guinea pigs, monkeys.

The term “target,” “target molecule,” or “target cell” refers to a molecule or biological cell of interest that is to be analyzed or detected, e.g., a ligand such as a cytokine or hormone, a polypeptide, a cellular receptor or a cell.

A cell has been “transformed” by exogenous or heterologous polynucleotide when such polynucleotide has been introduced inside the cell. The transforming DNA may or may not be integrated (covalently linked) into the genome of the cell. In prokaryotes, yeast, and mammalian cells for example, the transforming polynucleotide may be maintained on an episomal element such as a plasmid. With respect to eukaryotic cells, a stably transformed cell is one in which the transforming polynucleotide has become integrated into a chromosome so that it is inherited by daughter cells through chromosome replication. This stability is demonstrated by the ability of the eukaryotic cell to establish cell lines or clones comprised of a population of daughter cells containing the transforming polynucleotide. A “clone” is a population of cells derived from a single cell or common ancestor by mitosis. A “cell line” is a clone of a primary cell that is capable of stable growth in vitro for many generations.

The term “treating” or “alleviating” includes the administration of compounds or agents to a subject to prevent or delay the onset of the symptoms, complications, or biochemical indicia of a disease (e.g., Alzheimer's disease), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of a disease or disorder associated with or mediated by brain injury or neurodegeneration, a therapeutic agent may directly decrease the pathology of the disease, or render the disease more susceptible to treatment by other therapeutic agents.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

### III. In Vivo Migration Based Selection Format

The invention provides methods for identifying modulating agents that can induce a change in or differentiation of a population of a target cell, as well as migration of the differentiated or altered target cell to a specific location in the body of a human or a non-human animal (e.g., mice). In some embodiments, the type of the employed target cell is a stem cell type. In some of these embodiments, the employed candidate modulating agents are a combinatorial library of antibodies that are expressed in or introduced into the stem cells in vitro. Preferably, the stem cells employed for expressing the combinatorial antibody library are obtained from the same or same type of non-human animal that is used for the in vivo selection. Thus, in some embodiments, the stem cells (e.g., bone marrow cells) are modified ex vivo to express a combinatorial library of candidate agents (e.g., antibodies or peptides). The library of engineered stem cells are then reintroduced into the animal. After a sufficient period of time to allow cell differentiation and migration, one or more organs or tissues of interest (e.g., brain as exemplified herein) from the animal are examined for a desired phenotype and/or the presence of a specific candidate agent.

Methods of the invention can be employed for selecting modulating agents that promote cellular differentiation and migration into various organs or tissues of the body. Suitable organs or tissues where the differentiated agent-expressing cells may migrate into include those in, e.g., the musculoskeletal system (e.g., joints and ligaments), the digestive system (e.g., stomach, small intestine, liver and pancreas), the respiratory system (e.g., bronchi and lung), the cardiovascular system (e.g., heart and blood vessels), the immune system (e.g., spleen and lymph nodes), the nervous system (e.g., cerebellum, spinal cord, nerves), sensory organs (e.g., eye cornea, retina and ear organ components), and skin (e.g., subcutaneous tissue). In line with the diverse organs in the body, the cells in which to detect the presence and expressing of the modulating agents (e.g., antibodies) can be any of the diverse arrays of functional cells that are present in the different organs or tissues. Examples include, e.g., epithelial cells (such as exocrine secretory epithelial cells or keratinizing epithelial cells), sensory transducer cells (such as autonomic neuron cells, peripheral neuron supporting cells, central nervous system neurons and glial cells), and metabolism and storage cells (e.g., lung, gut, kidney, exocrine glands and urogenital tract cells, extracellular matrix cells, contractile cells, blood and immune system cells and interstitial cells).

As described below, the migration based in vivo selection methods of the invention can utilize a combinatorial antibody library (e.g., intracellularly expressed antibody library). In some embodiments, the antibody library is expressed with a lentiviral vector. As exemplified herein, a naïve human combinatorial antibody library (up to 1×10¹¹ library diversity) can be expressed from a lentiviral vector as scFv molecules. Viruses expressing the antibodies can be produced in an appropriate host cell, e.g., HEK293T cells. The heterogeneous population of antibody-expressing viruses can then be used to infect a population of stem cells (e.g., bone marrow cells) obtained from a non-human animal such as a mouse. To ensure efficient viral transduction, the bone marrow cells may be transduced with the lentiviral antibody library at a multiplicity of infection (MOI) of 2 or higher. The virus-bearing bone marrow cells are then transplanted to the animal for in vive selection. Typically, to facilitate subsequent differentiation and migration of the ex vivo modified bone marrow cells, the animal is lethally irradiated prior to transplantation. The animal with transplanted bone marrow cells can be maintained for an appropriate period of time to allow the stem cell differentiation and migration into the desired target organ or tissue in the body of the animal. Depending on the specific non-human animal employed in the selection and the target location, the period can be from several hours to several weeks. In some embodiments, a transplanted mouse can be maintained for a period of about 2 days to about 4 weeks, e.g., 3, 4, 5, 6, 7, 8, 9, 10, 11, 12, 13, 14, 16, 18, 21, 24, 28 days or even longer before the animal is examined for antibody expression at the target location (tissue or organ).

Many non-human animals can be employed in the in vivo selection methods of the invention. These include, e.g., any of the non-human mammals that have been used experimentally in the art. For example, the selection methods of the invention can use non-human animals such as cows, horses, sheep, pigs, cats, dogs, mice, rats, rabbits, guinea pigs, and monkeys. In some preferred embodiments, the employed non-human animal is a mouse. As exemplified herein, mice are used for selection of antibodies that promote stem cell differentiation into microglia and migration into the brain. In these embodiments, mice with transplanted antibody-expressing stem cells can be maintained for about 1-2 weeks. Thereafter, brain tissues from the mice can be perfused and harvested, followed by isolation of antibody-expressing cells or tissues. To facilitate isolation of antibody-expressing cells, the scFv antibodies expressed from the lentiviral vectors can be labeled or tagged. For example, the antibodies can be expressed as scFv-Fc tag fusions. The specific antibody encoding sequences can then be amplified and further analyzed. In addition, phenotype of the differentiated cells can also be examined to confirm activities of the selected antibody. As exemplification, antibody induced microglia can be examined by immunohistochemistry analysis to detect markers for microglia in brain sections of the transplanted mice.

In some embodiments, upon identifying antibodies with the migration based selection method, functional properties of the antibodies can be further examined and confirmed in vitro using purified antibodies. For example, a selected antibody can be examined in vitro to identify its target in the bone marrow cell. This can be accomplished via various assays that are routinely practiced in the art, e.g., immunoprecipitation, mass spectrometry and Western blot analysis. As exemplified herein, antibody B1 was found to recognize specific peptide sequences in the human VIM protein, e.g., SEQ ID NOs:25 and 26. In some embodiments, the selected antibody can be further examined in vitro to confirm its phenotype-inducing function observed in vivo. Thus, a microglia-inducing antibody can be examined for ability to promote stem cell differentiation into microglial cells. In this analysis, both human stem cells (e.g., human CD34+ cells) and mouse bone marrow cells can be used. As exemplified herein, phenotype of cells induced by the selected antibody in vitro can be analyzed by standard methods well known in the art. These include, e.g., flow cytometry and cell sorting (with antibodies recognizing appropriate cell surface markers), immunohistochemistry and immunofluorescent confocal microscopy. As exemplified herein, human CD34⁺ stem cells treated by the selected antibody can be examined via immunofluorescent staining. In vitro microglia-inducing activities of the selected antibodies on stem cells can also be examined with standard phagocytosis assay as exemplified herein.

### IV. Expressing Combinatorial Antibody Library

The in vive selection methods of the invention rely on expression of a combinatorial library of candidate agents (e.g., antibodies or peptides) in a population of stem cells (e.g., hematopoictic stem cells or embryonic stem cells). In some preferred embodiments, the library of candidate agents are a combinatorial library of antibodies. The combinatorial antibody library can be constructed (e.g., via lentiviral vectors) to provide efficient expression of antibodies upon introducing into the stem cells. The cellularly expressed antibodies can be secreted from or remain inside the cells to enable modulation of various targets of the stem cells. In some preferred embodiments of the in vive selection methods, the expressed antibodies are membrane-bound or remain inside the cells. Typically, to directly correlate an observed phenotype alteration with a specific antibody molecule or antibody-encoding sequence, the antibody library is introduced into and expressed in the cells under conditions each cell expresses no more than about 2 or 3 different antibodies or antibody-encoding sequences (e.g., scFv sequences). In some embodiments, each individual cell of the heterogeneous population of recombinantly produced cells expresses no more than one different member of the antibody library. With a lentiviral or retroviral based vector system as exemplified herein, this can be accomplished by infecting the producer or indicator cells the antibody-expressing viruses at a relatively low multiplicity of infection (MOI), e.g., not higher than 2 or 3. Under these conditions, an antibody modulator may be directly identified from an observed phenotype alteration with little or no further test of the antibodies that are isolated from cells at the target sites.

Any stem cells that are capable of differentiate into specific tissue or cell types can be used for expressing the combinatorial antibody library. These include embryonic stem cells, induced pluripotent stem (IPS) cells, hematopoietic stem cells (HSCs), and other tissue specific stem cells. In some embodiments, the stem cells employed for practicing methods of the invention are hematopoietic stem cells (HSCs). HSCs used in the invention can be obtained from a biological sample, e.g., bone marrow, of a human or a non-human animal (e.g., mice). Once expression vectors (e.g., lentiviral vectors) are introduced into the stem cells, the heterogeneous population of modified cells can be then introduced into the body (e.g., a non-human animal) for in vivo migration based selection.

The antibody library can express intact full length antibodies or antigen-binding fragments containing the antigen-binding portions of an intact antibody (i.e., antibody fragments that retain capacity to bind the cognate antigen). The antibodies produced by the antibody library can be single or double chain. In some embodiments, a single chain antibody library is expressed inside a eukaryotic producer cell. Single chain antibody libraries can comprise the heavy or light chain of an antibody alone or the variable domain thereof. More typically, members of single-chain antibody libraries are generated by a fusion of heavy and light chain variable domains separated by a suitable spacer within a single contiguous protein. See e.g., Ladner et al., WO 88/06630; McCafferty et al., WO 92/01047. In other embodiments, double-chain antibodies may be formed inside the producer cell by noncovalent association of separately expressed heavy and light chains or binding fragments thereof. The diversity of antibody libraries can arise from obtaining antibody-encoding sequences fiom a natural source, such as a non-clonal population of immunized or unimmunized B cells. Alternatively, or additionally, diversity can be introduced by artificial mutagenesis of antibodies for a target molecule. Typically, antibody libraries employed in the present invention contains at least 10², 10³, 10⁴, 10⁵, 10⁶, 10⁷, 10⁸, 10⁹, 10¹⁰ or more different members or species.

Various known libraries of antibodies can be utilized and modified as necessary in the practice of the selection methods of the invention. The antibody library can comprise unrelated antibodies from a naïve antibody library. For example, libraries of naïve antibodies (e.g., scFv libraries from human spleen cells) can be prepared as described in Feldhaus et al., Nat. Biotechnol. 21:163-170, 2003; and Lee et al., Biochem. Biophys. Res. Commun. 346:896-903, 2006. Park et al. (Antiviral Res. 68:109-15, 2005) also described a large non-immunized human phage antibody library in single-chain variable region fragment (scFv) format. Antibodies library derived from a subject with a specific disease can be prepared from RNA extracted from peripheral blood lymphocytes of the subject, using methods as described in Kausmally et al. (J. Gen. Virol. 85:3493-500, 2004). Alternatively, the antibody library can comprise synthetic antibodies or antibodies derived from a specific antibody, e.g., by DNA shuffling or mutagenesis. For example, Griffiths et al. (EMBO J 13:3245-3260, 1994) described a library of human antibodies generated from large synthetic repertoires (lox library). Some embodiments of the invention can employ libraries of antibodies that are derived from a specific scaffold antibody. Such antibody libraries can be produced by recombinant manipulation of the reference antibody using methods described herein or otherwise well known in the art. For example, Persson et al. (J. Mol. Biol. 357:607-20, 2006) described the construction of a focused antibody library for improved hapten recognition based on a known hapten-specific scFv.

In some preferred embodiments of the invention, the antibody library expresses single chain antibodies such as single chain variable region fragments (scFv). A specific scFv library suitable for the present invention is described in the Examples below and also in the art, e.g., Gao et al., Proc. Natl. Acad. Sci. 99:12612-6, 2002; and Zhang et al., PNAS 109:15728, 2012. Such an antibody library can be generated with and expressed from various vectors well known in the art. Preferably, the antibody library used in the invention is constructed via a lentiviral or retroviral based vector. Construction of such antibody library for expression inside a eukaryotic host cell can be performed in accordance with the techniques exemplified herein and other methods well known in the art. In some embodiments, the antibody library is constructed with a lentiviral vector. Lentiviral vectors are retroviral vectors that are able to transduce or infect both dividing and non-dividing cells and typically produce high viral titers. Examples of lentiviral based vectors suitable for the invention include, e.g., lentiviral vector pLV2. Other lentiviral vectors that may be employed and modified for practicing the invention include, e.g., pLVX-Puro, pLVX-IRES-Neo, pLVX-IRES-Hyg, and pLVX-IRES-Puro. The various lentiviral vectors with cloned antibody sequences can be introduced into an appropriate host cell for expressing the antibody library. For example, the HEK293T cell line exemplified herein, as well as other packaging cell lines well known in the art (e.g., Lenti-X 293T cell line), may be employed for expressing the antibody library in the invention. In addition to lentiviral based vectors and host cells, other retroviral based vectors and expression systems may also be employed in the practice of the methods of the invention. These include MMLV based vectors pQCXN, pQCXIQ and pQCXIH, and compatible producer cell lines such as HEK 293 based packaging cell lines GP2-293, EcoPack 2-293 and AmphoPack 293, as well as NIH/3T3-based packaging cell line RetroPack PT67.

### V. Antibodies Inducing Differentiation and Migration of Microglia

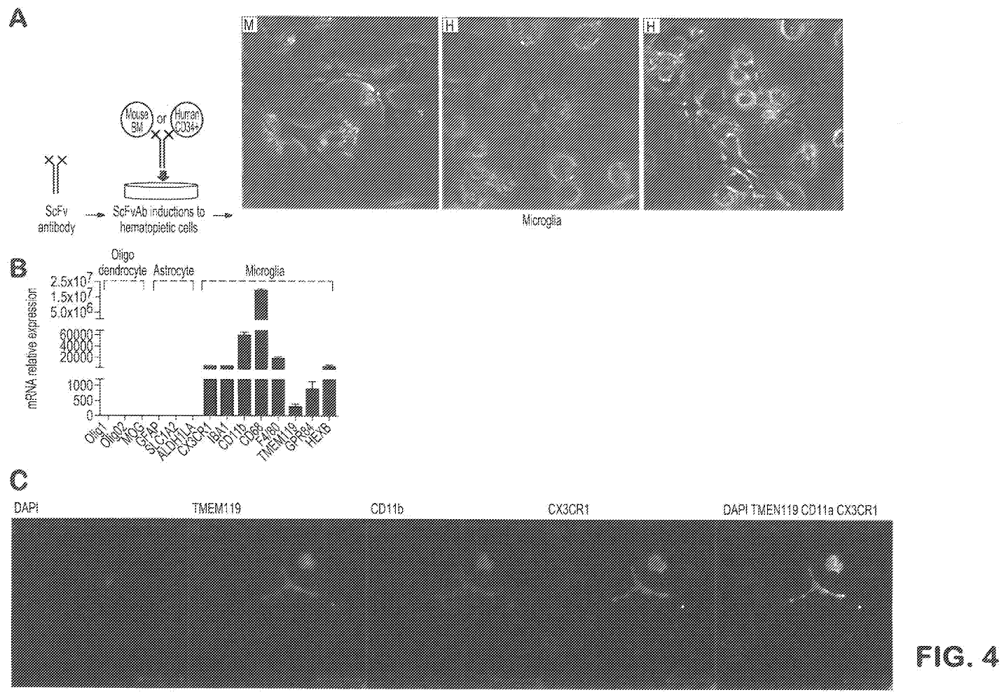

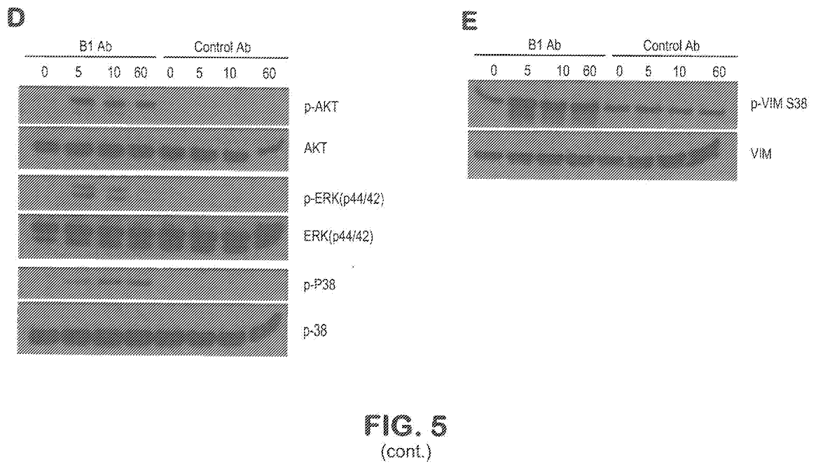

The invention provides novel antibodies that can induce stem cell differentiation into microglia and migration into the brain, which are also termed “microglia-inducing antibodies” herein. As exemplified herein, the inventors demonstrated that antibody B1 or B16 can induce microglia formation from both human and mouse bone marrow cells (see, e.g., FIGS. 3 and 5). Amino acid sequences of these two scFv antibodies are shown in SEQ ID NO:1 and 10, respectively. Additionally, it was identified that antibody B1 specifically binds to vimentin (VIM). The sequences of the heavy chain and the light chain portions of the B1 antibody are respectively shown in SEQ ID NOs:2 and 3. The CDR sequences of the heavy chain variable region of this antibody are GFNFNNYN (SEQ ID NO:4), ISSAADTV (SEQ ID NO:5), ARQLLY (SEQ ID NO:6). The CDR sequences of its light chain variable region are SGINVGAYR (SEQ ID NO:7), YKSDSDK (SEQ ID NO:8), and AIWHSSAWV (SEQ ID NO:9). Similarly, the amino acid sequences of the heavy chain and the light chain portions of the B16 antibody are respectively shown in SEQ ID NOs:11 and 12. Its annotated CDR sequences in the heavy chain are GFTFSSYA (SEQ ID NO:13), MSGSGGST (SEQ ID NO:14), AKGVWFGELLPPFDY (SEQ ID NO:15).

Typically, the antibodies or antigen-binding fragments of the invention have the same binding specificity as that of a reference antibody that is derived from an antibody exemplified herein (e.g., antibody B1 or B16). In various embodiments, the reference antibody has (a) heavy chain CDRs 1-3 and light chain CDRs 1-3 sequences that are respectively identical to (1) GFN FNNYN (SEQ ID NO:4), ISSAADTV (SEQ ID NO:5), ARQLLY (SEQ ID NO:6), SGIN VGAYR (SEQ ID NO:7), YKSDSDK (SEQ ID NO:8), and AIWHSSAWV (SEQ ID NO:9), (b) heavy chain and light chain variable region sequences respectively shown in SEQ ID NOs:2 and 3, or (c) heavy chain and light chain variable region sequences respectively shown in SEQ ID NOs:11 and 12. The antibodies of the invention for inducing microglia are preferably monoclonal antibodies like the antibodies exemplified in the Examples below. In some embodiments, the antibodies have the same binding specificity as that of the B1 or B16 agonist antibody. In some embodiments, the antibodies of the invention compete with antibody B for binding to the specific VIM peptides recognized by antibody B1 (e.g., peptides shown in SEQ ID NO:25 or 26). In addition to containing variable regions sequences derived from the B1 or B16 antibody, some microglia-inducing antibodies of the invention can also contain other antibody sequences fused to the variable region sequences. For example, the antibodies can contain an Fc portion of IgG. The antibodies can also be conjugated, covalently or noncovalently, to another entity that specifically targets a surface antigen, receptor or marker on target cells, e.g., stem cells, bone marrow cells, or mesenchymal cells.

In some embodiments, the microglia inducing antibodies or antigen-binding fragments of the invention have heavy chain CDRs 1-3 sequences that are substantially identical, respectively, to (1) GFN FNNYN (SEQ ID NO:4), ISSAADTV (SEQ ID NO:5), ARQLLY (SEQ ID NO:6) or (2) GFTFSSYA (SEQ ID NO:13), MSGSGGST (SEQ ID NO:14), AKGVWFGELLPPFDY (SEQ ID NO:15). Some of these antibodies or antigen-binding fragments can additionally contain light chain CDRs 1-3 sequences that are substantially identical, respectively, to SEQ ID NOs:7-9. Some antibodies or antigen-binding fragments of the invention contain a heavy chain CDR sequence selected from the group consisting of SEQ ID NOs:4-6 and 13-15. Some of these antibodies can additionally contain a light chain CDR sequence selected from the group consisting of SEQ ID NOs:7-9. Some of these antibodies or antigen-binding fragments of the invention have heavy chain CDRs 1-3 sequences that are respectively identical to (1) SEQ ID NOs:4-6 or (2) SEQ ID NOs: 13-15. Some of these antibodies or antigen-binding fragments have heavy chain CDRs 1-3 and light chain CDRs 1-3 sequences respectively shown in SEQ ID NOs:4-6 and SEQ ID NOs:7-9.

In some embodiments, the invention provides antibodies or antigen-binding fragments that are conservatively modified variants of the antibodies exemplified herein. Typically, the variable regions of these variants have an amino acid sequence that is identical to one of these exemplified sequences (e.g., SEQ ID NOs:2, 3, 11 and 12) except for conservative substitution at one or more amino acid residues. In some of these embodiments, the antibodies or antigen-binding fragments have heavy chain CDRs 1-3 and light chain CDRs 1-3 sequences respectively shown in SEQ ID NOs:4-6 and SEQ ID NOs:7-9, except for conservative substitution at one or more amino acid residues in the CDRs.

Some microglia inducing antibodies or antigen-binding fragments of the invention have a light chain CDR sequence selected from the group consisting of SEQ ID NOs:7-9. Some of these molecules can additionally contain a heavy chain CDR sequence selected from the group consisting of SEQ ID NOs:4-6 and 13-15. Some of these molecules have light chain CDRs 1-3 sequences that are respectively identical to SEQ ID NOs:7-9. Some of the microglia inducing antibodies or antigen-binding fragments of the invention have a heavy chain variable region sequence that is substantially identical to SEQ ID NO:2 or 11. Some microglia inducing antibodies or antigen-binding fragments of the invention have a light chain variable region sequence that is substantially identical to SEQ ID NO:3 or 12. Some antibodies or antigen-binding fragments of the invention have a heavy chain variable region sequence and a light chain variable region sequence that are substantially identical, respectively, to (1) SEQ ID NO:2 and 3 or (2) SEQ ID NO:11 and 12. In some embodiments, the microglia inducing antibodies or antigen-binding fragments of the invention contain a heavy chain variable region sequence and a light chain variable region sequence, one or both of which are identical to a heavy chain variable region sequence and a light chain variable region sequence respectively shown in (1) SEQ ID NOs:2 and 3 or (2) SEQ ID NOs:11 and 12. Some of these molecules have a heavy chain variable region sequence and a light chain variable region sequence respectively shown in (1) SEQ ID NOs:2 and 3 or (2) SEQ ID NOs:11 and 12.

In various embodiments, the antibodies or antigen-binding fragments of the invention can be IgA1, IgA2, IgD, IgE, IgG1, IgG2, IgG3, IgG4, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, and a bivalent antibody. In some embodiments, the molecule is an IgG selected from the group consisting of IgG1, IgG2, IgG3, IgG4, and synthetic IgG. In some embodiments, the molecule is a Fab, a scFv, or a dsFv. In some preferred embodiments, the microglia-inducing antibodies of the invention are scFv fragments as exemplified herein in SEQ ID NO:1 or 10. In some scFv fragments of the invention, the heavy chain and light chain variable region sequences are connected via a linker sequence. For example, the variable region sequences can be connected with a linker sequence GGGGGS (SEQ ID NO:16) or GGGGSGGGGSGGGGS (SEQ ID NO:17). In some embodiments, an antibody or antigen-binding fragment of the invention can be further conjugated to a synthetic molecule such as a marker or detectable moiety.

Some microglia-inducing agonist antibodies of the invention harbor variable region sequences that are substantially identical (e.g., at least 90% or 95% identical) to that of the B1 or B16 antibody. Some other microglia-inducing antibodies have all CDR sequences in their variable regions of the heavy chain and light chain that are respectively identical or substantially identical (e.g., at least 90% or 95% identical) to the corresponding CDR sequences of the B1 or B16 agonist antibody. In still some other embodiments, the microglia-inducing antibody has its entire heavy chain and light chain variable region sequences respectively identical to the corresponding variable region sequences of the B1 or B16 antibody. In some other embodiments, other than the identical CDR sequences, the antibodies contain amino acid residues in the framework portions of the variable regions that are different from the corresponding amino acid residues of the B1 or B16 antibody. Relative to the B1 or B16 antibody, the agonist antibodies of the invention can undergo non-critical amino-acid substitutions, additions or deletions in the variable region without loss of binding specificity or effector functions, or other modifications that do not cause intolerable reduction of binding affinity for the target antigen (e.g., VIM-peptides disclosed herein). Usually, antibodies incorporating such alterations exhibit substantial sequence identity to the B1 or B16 antibody. For example, the mature light chain variable regions of some of the agonist antibodies of the invention have at least 75%, at least 85% or at least 90% sequence identity to the sequence of the mature light chain variable region of the B1 or B16 antibody. Similarly, the mature heavy chain variable regions of the antibodies typically show at least 75%, at least 85% or at least 90% sequence identity to the sequence of the mature heavy chain variable region of the B1 or B16 antibody. In various embodiments, the antibodies typically have their entire variable region sequences of the heavy chain and/or light chain that are substantial identical (e.g., at least 75%, 85%, 90%, 95%, or 99%) to the corresponding variable region sequences of the B1 or B16 antibody. Some Microglia-inducing agonist antibodies of the invention have the same binding specificity but improved affinity activities if compared with the B1 or B16 antibody.

The microglia-inducing antibodies of the invention can be generated in accordance with routinely practiced immunology methods. Some of such methods are exemplified herein in the Examples. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, N.Y., 1998; Kohler & Milstein, Nature 256:495-497, 1975; Kozbor et al., Immunology Today 4:72, 1983; and Cole et al. pp. 77-96 in Monoclonal Antibodies and Cancer Therapy, 1985.

### VI. Polynucleotides, Vectors and Host Cells for Producing Microglia-Inducing Antibodies

The invention provides substantially purified polynucleotides (DNA or RNA) which encode polypeptides comprising segments or domains of the microglia-inducing antibody chains or antigen-binding molecules described herein. Some of the polynucleotides of the invention contain a nucleotide sequence that encodes the scFv antibody fragment sequence as shown in SEQ ID NO:1 or 10. Some of the polynucleotides of the invention contain a nucleotide sequence that encodes the heavy chain variable region as shown in SEQ ID NO:2 or 11 and/or the light chain variable region sequence as shown in SEQ ID NO:3 or 12. Also provided in the invention are polynucleotides which encode at least one CDR region and usually all three CDR regions from the heavy or light chain of the antibodies described herein. Because of the degeneracy of the code, a variety of nucleic acid sequences will encode each of the exemplified amino acid sequences. Some other polynucleotides of the invention comprise nucleotide sequences that are substantially identical (e.g., at least 65%, 80%, 95%, or 99%) to one of the nucleotide sequences shown in SEQ ID NOs: 18-23. When expressed from appropriate expression vectors, polypeptides encoded by these polynucleotides are capable of exhibiting antigen binding capacity.

In some embodiments, the polynucleotides of the invention can encode only the variable region sequence of a microglia inducing antibody. They can also encode both a variable region and a constant region of the antibody. Some of polynucleotide sequences of the invention nucleic acids encode a mature heavy chain variable region sequence that is substantially identical (e.g., at least 80%, 90%, or 99%) to the mature heavy chain variable region sequence shown in SEQ ID NO:2 or 11. Some other polynucleotide sequences encode a mature light chain variable region sequence that is substantially identical to the mature light chain variable region sequence shown in SEQ ID NO:3 or 12. Some of the polynucleotide sequences encode a polypeptide that comprises variable regions of both the heavy chain and the light chain of one of the exemplified antibody. Some other polynucleotides encode two polypeptide segments that respectively are substantially identical to the variable regions of the heavy chain and the light chain of one of the exemplified antibodies (e.g., antibody B1 or B16).

The polynucleotide sequences can be produced by de novo solid-phase DNA synthesis or by PCR mutagenesis of an existing sequence (e.g., sequences as described in the Examples below) encoding a microglia-inducing antibody or antigen-binding fragment. Direct chemical synthesis of nucleic acids can be accomplished by methods known in the art, such as the phosphotriester method of Narang et al., Meth. Enzymol. 68:90, 1979; the phosphodiester method of Brown ct al., Meth. Enzymol. 68:109, 1979; the diethyiphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

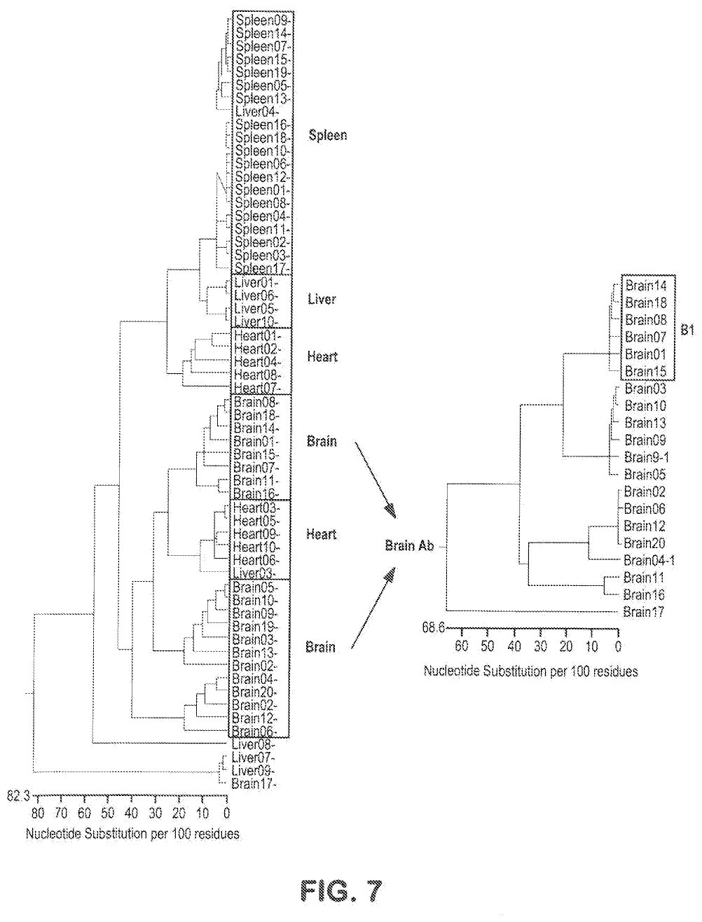

Also provided in the invention are expression vectors and host cells for producing the antibodies described herein. Specific examples of lentiviral based vectors for expressing the antibodies are described in the Examples below (see FIG. 7). Various other expression vectors can also be employed to express the polynucleotides encoding the microglia-inducing antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the polynucleotides and polypeptides of the invention in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on lentiviruses or other retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a microglia-inducing antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a microglia-inducing antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted microglia-inducing antibody sequences. More often, the inserted microglia-inducing antibody sequences are linked to a signal sequences before inclusion in the vector. Vectors to be used to receive sequences encoding the microglia-inducing antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the microglia-inducing antibody chains can be either prokaryotic or eukaryotic. In some preferred embodiments, mammalian host cells are used to express and produce the antibody polypeptides of the present invention. For example, they can be either a hybridoma cell line expressing endogenous immunoglobulin genes or a mammalian cell line harboring an exogenous expression vector (e.g., the HEK293T cells exemplified below). These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

### VII. Therapeutic Applications

In some embodiments, the invention provides methods for producing functional microglial cells in vitro from stem cells. In these methods, a stem cell population such as bone marrow cells or HSCs can be contacted with a microglia-inducing antibody described herein (e.g., antibody B1 or B16) and cultured in vitro under appropriate condition. Differentiation of the stem cells into microglial cells can be monitored and examined by detecting one or more microglial cell markers. Detailed procedures for culturing stem cells in the presence of the antibody and for assessing phenotype of the differentiated cells are exemplified herein (e.g., Examples 3 and 5-7 below). The invention additionally provides kits or pharmaceutical combinations for converting HSCs or bone marrow cells into microglial cells. The kits typically contain one or more microglia-inducing antibodies described herein, tools and materials for isolating bone marrow cells or HSCs from a subject, and reagents for co-culturing the cells with the agonist antibody. In some embodiments, the kits can contain the agonist antibody and a cultured bone marrow cell population for generating microglia that can be applied allogeneically to subjects afflicted with brain injuries or infections.

In some other embodiments, the invention provides therapeutic uses or methods of the described microglia inducing antibodies in treating brain disorders, injuries or infections. In some other embodiments, stem cells (e.g., bone marrow cells) are first treated with a microglia-inducing antibody exemplified herein, and the modified stem cell population is then administered to subjects in need of treatment for the noted brain disorders. In some of these methods, the stem cells are isolated from the subject in need of treatment. In some of these methods, the antibodies or cell populations are used to treat or ameliorate symptoms associated with chronic loss of memory and other mental abilities, e.g., dementia. Subjects afflicted with or at risk of developing various types of dementia or related disorders are all suitable for therapeutic or prophylactic treatment with the microglia inducing antibodies of the invention. Among these neurodegenerative disorders, Alzheimer's disease is the most common type of dementia and accounts for an estimated 60 to 80 percent of cases. Other disorders include, e.g., vascular dementia, dementia with Lewy bodies (DLB), mixed dementia, Parkinson's disease, frontotemporal dementia, Creutzfeldt-Jakob disease, normal pressure hydrocephalus, Huntington's disease, Wernicke-Korsakoff syndrome, mild cognitive impairment. AIDS dementia, Pick's disease, Nieman-Pick Disease, posterior cortical atrophy, progressive supranuclear palsy and Down's syndrome. In some methods, the antibodies may be employed in treating traumatic brain injury (TBI). In TBI, mechanical injury initiates cellular and biochemical changes that perpetuate neuronal injury and death over time, a process known as secondary injury. These include glutamate excitotoxicity, blood-brain barrier disruption, secondary hemorrhage, ischemia, mitochondrial dysfunction, apoptotic and necrotic cell death, and inflammation. As the primary mediators of the brain's innate immune response to infection, injury, and disease, microglia react to injury within minutes. Microglia can produce a number of neuroprotective substances after injury, including anti-inflammatory cytokines and neurotrophic factors, including nerve growth factor and transforming growth factor β (TGF-β). These neuroprotective effects may be a result of suppressed microglial production of proinflammatory cytokines.

The microglia inducing antibodies, or stem cell population treated thereby, of the invention can also be used in protecting the brain against infections. The blood-brain barrier prevents most infections from reaching the vulnerable nervous tissue in the brain. Nevertheless, when infectious agents are directly introduced to the brain or cross the blood-brain barrier, microglial cells must react quickly to decrease inflammation and destroy the infectious agents before they damage the sensitive neural tissue. Due to the unavailability of antibodies from the rest of the body (few antibodies are small enough to cross the blood-brain barrier), the body relies on microglia to recognize foreign bodies, swallow them, and act as antigen-presenting cells activating T-cells. Activated phagocytic microglia are the maximally immune responsive form of microglia. They travel to sites of the neuronal injury, engulf the offending material, and secrete pro-inflammatory factors to promote more cells to proliferate and do the same. Activated phagocytic microglia also interact with astrocytes and neural cells to fight off the infection as quickly as possible with minimal damage to the healthy brain cells.

In various therapeutic applications of the invention, subjects afflicted with a brain disorder (e.g., Alzheimer's disease) or infection can be administered with a microglia inducing antibody (e.g., antibody B1) that is capable of promoting stem cell differentiation into microglia and migration into the brain. Typically, a subject is administered a pharmaceutical composition that contains a therapeutically effective amount of a microglia inducing antibody or antibody treated stem cell population as disclosed herein. In some embodiments, a stem cell population (e.g., bone marrow cells) may be first treated in vitro with an agonist antibody described herein prior to being introduced into the body of the subject to promote microglia differentiation and migration into the brain. In some of these methods, the employed stem cells are human bone marrow cells. In some methods, the stem cell population is isolated from the same subject in need of treatment. In some embodiments, the cells are cultured with the antibody for about 4 to 20 days. Some of the methods can additionally include detecting in the cultured cell population at least one cellular marker expressed by microglial cells, e.g., CX3CR1, IBA1, CD1 lb, CD68, F4/80, TMEMI19, GPR84, and HEXB.

The pharmaceutical compositions containing a microglia-inducing antibody or a microglial cell population described herein can be administered to subjects in need of treatment in accordance with standard procedures of pharmacology. Methods of administering the therapeutic compositions to a subject can be accomplished based on procedures routinely practiced in the art. See, e.g., Remington: The Science and Practice of Pharmacy, Mack Publishing Co., 20^{th} ed., 2000; Ritter et al., J. Clin. Invest. 116:3266-76, 2006; Iwasaki et al., Jpn. J. Cancer Res. 88:861-6, 1997; Jespersen et al., Eur. Heart J. 11:269-74, 1990; and Martens, Resuscitation 27:177, 1994. For example, a composition containing the induced M2 macrophages are typically administered (e.g., via injection) in a physiologically tolerable medium, such as phosphate buffered saline (PBS). The isolated cells, or their engineered form as disclosed herein, should be administered to the subject in a number sufficient to inhibit the development of the disease in the subject. In some embodiments, administration of therapeutic composition is carried out by local or central injection of the cells into the subject. In some other embodiments, the administration is via a systemic route such as peripheral administration. Additional guidance for preparation and administration of the pharmaceutical compositions of the invention are described in the art. See, e.g., Goodman & Gilman's The Pharmacological Bases of Therapeutics, Hardman et al., eds., McGraw-Hill Professional (10a ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

### EXAMPLES

The following examples are provided to further illustrate the invention but not to limit its scope. Other variants of the invention will be readily apparent to one of ordinary skill in the art and are encompassed by the appended claims.

### Example 1 In Vive Selection of Antibodies that Regulate Migration

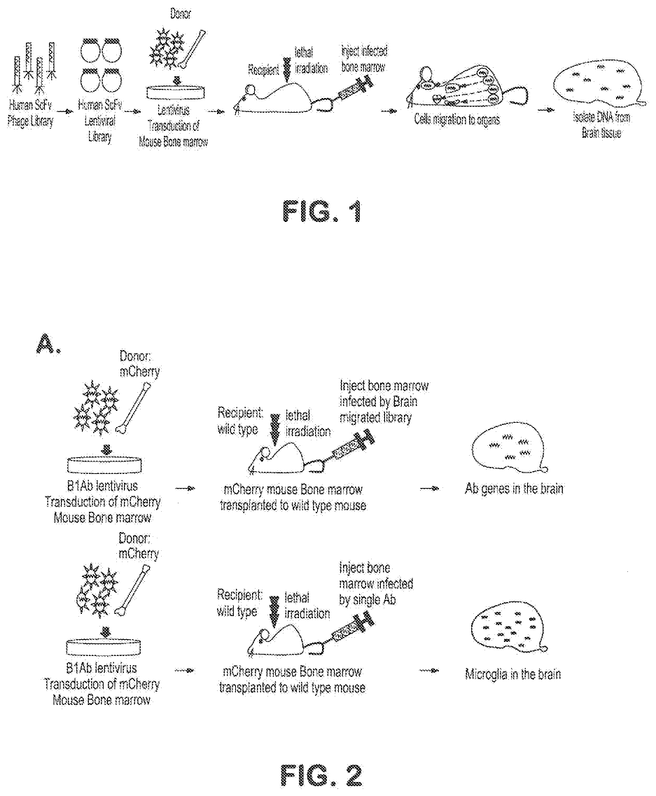

A novel in vivo selection scheme was developed (FIG. 1) to identify antibodies that cause differentiation of human stem cells (HSCs) into cell types capable of migration to specific tissues in the body such as the brain where migratory cells are thought to be important in Alzheimer's and Parkinson's disease. Genes obtained from a human short-chain fragment variable (ScFv) phage library were used to create a human ScFv lentiviral intracellular combinatorial antibody library, with 10⁸ unique antibody clones. In this system the antibodies were expressed on the cell surface using previously reported methodology (Xie et al., Proc. Nati. Acad. Sci. USA 110, 8099-8104, 2013). Total bone marrow cells were harvested from mice, infected in vitro with the lentiviral library, and then transplanted into lethally irradiated mice. After 7 days, brains of PBS perfused mice were harvested to extract genomic DNA which was subjected to PCR in order to amplify and sequence human scFv sequences that were integrated into the genome of migrating cells. Different organ systems were studied and each contained cells that had different antibody genes incorporated into their genome (FIG. 7).

### Example 2 Selected Antibody B1 Induces Migration of Cells to Brain

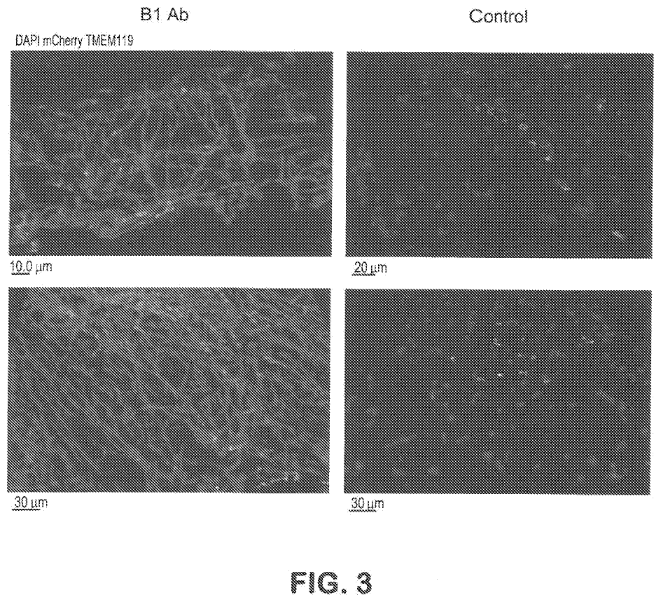

As a preliminary experiment to confirm that the integrated antibody genes induced bone marrow cells to migrate to the brain, the entire collection of antibody genes that were recovered from the migrating cell population were cloned into lentivirus vectors which were then inserted into the genomes of fresh bone marrow cells from mice expressing the red fluorescent protein (mCherry). We adoptively transferred these donor mCherry⁺ bone marrow cells that now contained the selected antibody genes into irradiated wild-type mice (FIG. 2). After two weeks, brains were perfused, harvested, and analyzed for the presence of cells ubiquitously expressing mCherry. The brains contained many cells expressing the mCherry marker, indicating that at least some members of the selected antibody population could induce cells to migrate to the brain. More details of this study are as follows: after 3 days, these cells were transplanted into lethally irradiated wild type C57BL6J mice. After 2 weeks and 1 week respectively, the mice were perfused with PBS followed by 2% PFA prior to harvesting the brains and sectioning the frozen OCT blocks for immunofluorescence histochemistry. Brain sections (10 μm) were stained with DAPI and anti-mCherry antibodies to amplify the signal and then analyzed by confocal microscopy. Further, mCherry⁺ cells were identified in the treated tissues as comparted to controls, suggesting that mCherry⁺ donor cells migrated from the bone marrow to the brain.

Two antibody genes, B1 and B16 were identified from the study. Nucleotide and amino acid sequences of the antibodies are shown below. In the sequences, underlined residues correspond to the heavy chain variable region, italicized residues are that of the linker sequence, and the remaining sequence is the light chain variable region. In addition, CDR motifs are double underlined in the sequences.

```
    1 ATGGCACAGG TGCAGCTGTT GGAGTCTGGG GGAGGTTTGG TGCAGCCGGG GGGGTCCCTG

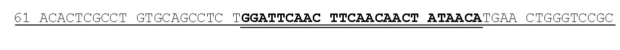

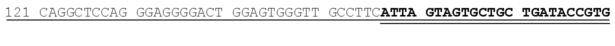


  181 CACTACGCAG ACTCTGTGGA GGGCCGATTC ACCATCTCCA GAGACAACGG CAAGAACTCA

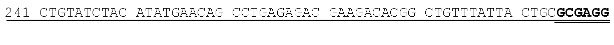

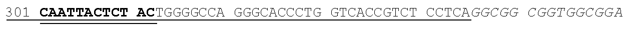


  361 TCCCAGGCTG TGCTCACTCA GCCGTCTTCC CTCTCTGCAT CTCCTGGAGC ATCAGTCAGT

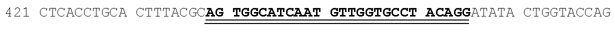

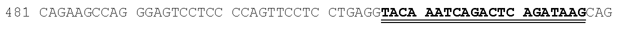


  541 CAGGGCTCTG GAGTCCCAG CCGCTTCTCT GGATCCAGAG ATGCTTCGGC CAATGCAGGG

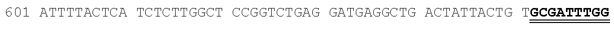

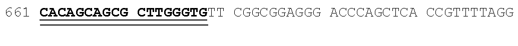

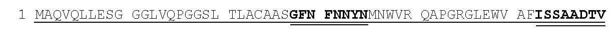

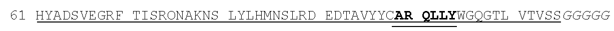

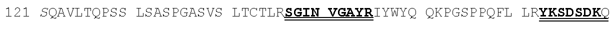

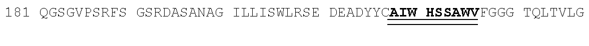






B16 antibody sequences (696 nucleotides, SEQ ID NO: 68; 232 amino acids,
SEQ ID NO: 10):
    1 ATGGCACAGG TGCAGCTGCA GGAGTCCGGG GGAGGCTTGG TACAGCCTGG GGGGTCCCTG

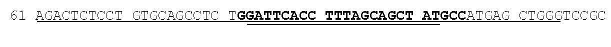

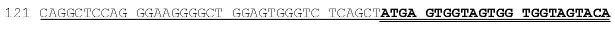


181 TACTACGCAG ACTCCGTGAA GGGCGGTTC ACCATCTCCA GAGACAATTC CAAGAACACG

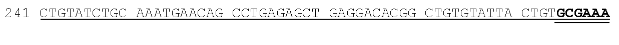

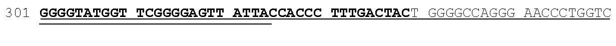


  361 ACCGTCTCCT CAGGCGGCGG CGGCTCTGGC GGAGGTGGCA GCGGCGGTGG CGGATCCAAT
  421 TTTATGCTGA CTCAGCCCCA TGACCCATGG CCCACAGCCC ACAGCACACA GGACACAGCA
  481 CACAGCACAC AGGACACAGC CCACAGCCCA CAGCACACAG GACACAGCAC ACAGCACACA
  541 GGACACAGCC CACAGCACAC AGGACACAGC ACACAGGACA CAGCCCACAG CACACAGGAC
  601 ACAGCCCACA GCACACAGCC CACAGCCCAC AGCACACAGG ACACAGCCCA CAGCCCACAG
  661 CACACAGGAC ACGGGACCCA GCTCACCGTT TTAGGT

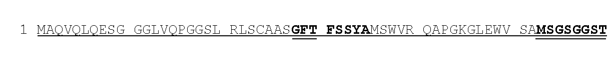

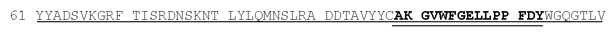


121 TVSSGGGGSG GGGSGGGGSN FMLTQPHDPW PTAHSTQDTA HSTQDTAHSP QHTGHSTQHT
181 GHSPQHTGHS TQDTAHSTQD TAHSTQPTAH STQDTAHSPQ HTGHGTQLTV LG
```

To investigate whether a single antibody could induce cell migration from the bone marrow to the brain, the B1 gene alone was cloned into a lentivirus vector that was used to infect total bone marrow cells that were freshly harvested from mice that ubiquitously expressed monomeric mCherry protein. The B1 gene was selected for study because of the four antibody gene sequences that were recovered from the cells that had migrated to the brain, it had the highest copy number (FIG. 7). In antibody selections, because the number of input sequences are so high, selection of repeated sequences is of special significance. These mCherry⁺ cells with the single lentivirus encoded B1 antibody integrated into their genome were then transplanted into lethally irradiated wild-type mice (FIG. 2A). After 1 week, brains were perfused and prepared for immunofluorescence histochemistry. Donor mCherry⁺ cells infected with the B1 Ab migrated to the brain (FIG. 2B) and also stained positive for the microglia marker TMEM119. A comprehensive analysis of whole brain sections from treated and control mice showed that a significantly greater mCherry⁺ signal (63,270 versus 4,104 fluorescent units) was detected in the hippocampus, substantia nigra, and hypothalamus in mice whose bone marrow was infected with lentivirus encoding the B1 Ab. Importantly, the migrating cells appear to organize themselves into extensive microglia networks as observed in the 3D images (FIG. 3).

As additional details for this study, a single gene (B1 Ab) was reinserted into a lentiviral vector and used to infect total mCherry⁺ mouse bone marrow cells. After 3 days, these cells were transplanted into lethally irradiated wild type C57BL/6J mice. After 2 weeks, the mice were perfused with PBS followed by 2% PFA prior to harvesting the brains for sectioning of the frozen OCT blocks for immunofluorescence histochemistry. Brain sections (10 μm) were stained with DAPI, mCherry antibody to amplify the mCherry signal, and the TMEM119 antibody to identify microglia. Sections were analyzed by confocal microscopy. mCherry⁺ cells were co-stained for the TMEM119 marker, suggesting the mCherry⁺ donor cells that migrated from the bone marrow to the brain were microglia. Fluorescent units of mCherry⁺ signal were quantified by imagePro software in whole brain sections of the same tissue by confocal microscopy on lower magnification. A mCherry⁺ signal was detected in the hippocampus, substantia nigra, and hypothalamus of B1 Ab-infected mice.

This in vivo bioluminescence imaging study confirmed that integrated antibody genes induced the migration of the bone marrow cells. To perform this study, fresh bone marrow cells from luciferase-expressing transgenic mice (luc⁺) were infected with lentivirus encoding the B1 Ab, adoptively transferred into irradiated wild-type mice, and imaged after one week. As indicated in the figure, the results indicate that donor luc⁺ cells infected with the B1 Ab migrated to the brain.

Currently it is thought that in adoptive transfer experiments involving irradiation, some white blood cells are able to migrate into the brain due to a compromised blood brain barrier as a result of inflammation caused by the irradiation. To study the role of irradiation, mice were irradiated with or without a lead helmet and analyzed two weeks later by histochemistry. No mCherry⁺ cells were seen in the brain when mice were wearing a lead helmet during irradiation (data not shown). This result is in agreement with the studies of others. In particular, it was previously shown that in adoptive transfer experiments when the brain was shielded from irradiation, significant invasion of bone marrow-derived microglia into the brain was not observed which was in contrast to the results in unshielded mice where significant invasion was seen (Mildner et al., Nat. Neurosci. 10, 1544-1553, 2007). The results presented here are the first report of a single agonist that induces microglia-like cells, which have the capacity to migrate to the brain.

### Example 3 Purified Antibody Differentiates Human and Murine Stem Cells into Microglia

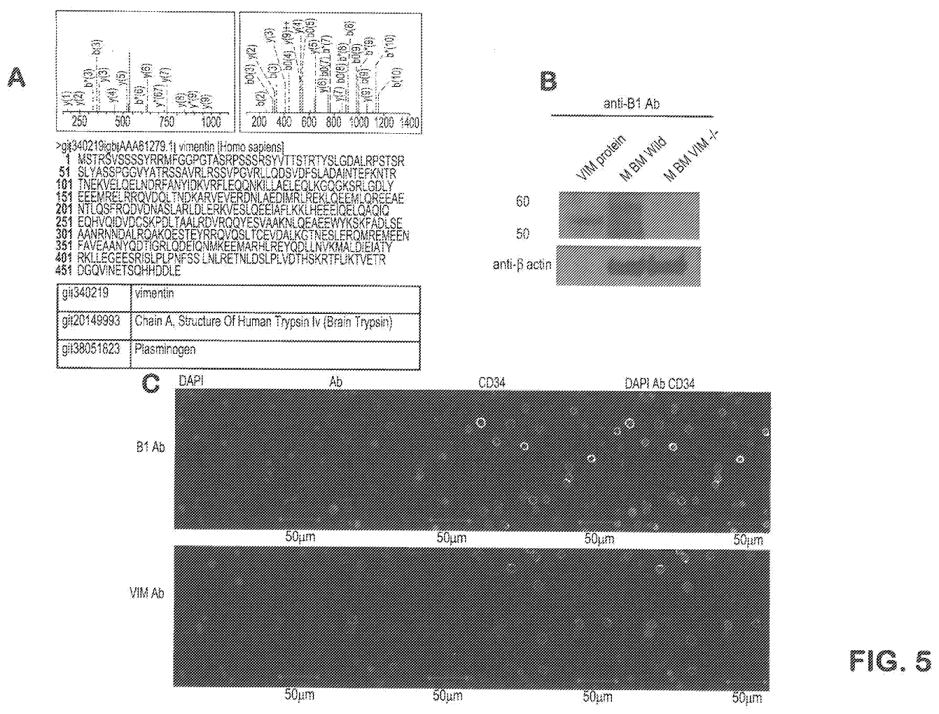

To determine if purified antibody as opposed to integrated lentivirus could transform bone marrow cells, total mouse bone marrow or human CD34⁺ cells were incubated with the selected B1 Ab (at a concentration of about 5-10 g/ml) for two weeks in vitro. The purified antibody induced both the mouse and human cells to differentiate into cells with a cellular morphology resembling microglia that had extensive branched processes (FIG. 4A). To study the nature of the induced cells, mRNA expression levels were analyzed for specific oligodendrocyte, astrocyte, and microglia marker genes by qRT-PCR. The cells induced by purified B1 Ab expressed mRNA for the microglial markers CX3CR1, IBA1, CD11b, CD68, F4/80, TMEM119, GPR84, and HEXB, but failed to express mRNA for the established oligodendrocyte (Olig1, Olig2, and MOG) and astrocyte (GFAP, SLC1A2, and ALDH1LA) gene markers (FIG. 4B). Immunofluorescent-cytochemical analysis of B1 Ab-differentiated human CD34 cells, using the microglia specific markers TMEM119, CD11b, and CX3CR1 provided further evidence that the differentiated cells had staining patterns of microglia (FIG. 4C).

RNA transcripts of human CD34⁺ cells treated with purified B1 Ab were also sequenced and compared to the profile of macrophages induced by treatment of human CD34⁺ cells with M-CSF in vitro. RNA sequencing data from human CD34⁺ cells treated with B1 Ab or M-CSF were consistent with qRT-PCR results. To further identify transcripts that are expressed in microglia, we compared the results to expression data of previous reports (D. Gosselin et al., Science, 356:1248, 2017 and J. Muffat et al, Nat Med, 22:1358-1367, 2016). Notably, we found genes highly expressed in microglia, which include IGTAM, IBA1, TREM2, APOE, CD33, ITGB2, ADORA3, LGMN, PROS 1, C1QA, GPR34, TGFBR1, SELPLG, HEXB, LTC4S, and CCL2 to be consistent with data published by other groups. Importantly, we also found B1 Ab induced microglia have a gene expression similar to human microglia. Among 52 genes the most highly expressed are from human microglia [75% of the genes (39/52)] which is consistent with our data.

To classify similarities and differences between the induced microglia and macrophages, we compared the top 10% of transcripts with the highest expression levels. Of the 3,996 total transcripts identified, 3,098 transcripts were shared between microglia and macrophages, 243 were unique to microglia differentiated with B1Ab, and 312 were unique to macrophages differentiated with MCSF. The most highly expressed genes that were expressed in both microglia and macrophages were ACP5, MMP9, APOCI, CTSL, COL6A2, CTSK, CYP27A1, and MSRI. The highly expressed genes unique to microglia included RPL3P4, FBPI, LIF, IL9R, SIGLEC6, MARCO, UTS2, CKAP4, and GPRC5C, whereas genes uniquely expressed in macrophages included RNASE1, LAIR2, PFKFB3, RNASE6, and GPR183. Of the highly expressed genes specific to microglia, 268 have been reported to be relevant to neuronal diseases such as Alzheimer's, amyloidosis, tauopathy, dementia, inflammation of central nervous system, and encephalitis.

### Example 4 Identification of Target of Antibody B1

To identify the protein recognized by the B1 antibody, antibodies were produced recombinantly in Expi293F cells. Purified B1 antibody (at a concentration of about 5-10 μg/ml) was incubated with human CD34⁺ cells, and immune complexes from cellular lysates were captured on a protein A/G column. Proteins that reacted with the antibody were identified by silver staining of SDS gels and their identity determined by mass spectrometry (MS). Three candidate proteins were identified above the background threshold (FIG. 5A). Vimentin (VIM) was one of the top hits and was confirmed to be B1 target antigen of B1 Ab by Western blotting. The B1 Ab bound to purified VIM protein as well as VIM from wild-type mouse bone marrow lysates, but did not bind to proteins in lysates from bone marrow obtained from VIM-deficient knock out mice (FIG. 5B). Also, VIM expression was found in human CD34⁺ cells by immunofluorescence cytochemistry using B1 or commercial VIM antibodies (FIG. 5C). The amino acid sequence identity between mouse and human VIM is greater than 97%.

In further studies, bone marrow from wild type and VIM knockout mice were incubated with B1 Ab or commercial VIM Ab for 6 days. FACS analysis showed that microglia formation was increased by B1Ab in wildtype mice. However, there was no induction of microglia in the VIM knockout mice. Interestingly, commercial VIM Ab didn't induce microglia differentiation, indicating that our antibody had a unique binding mode because it was the product of selection for migration rather than simple binding.

### Example 5 Selected Antibody B1 Induces a Signal Transduction Cascade

To determine whether binding of B1 Ab leads to the activation of signaling pathways, human bone marrow CD34⁺ cells were treated with the B1 Ab, and cell lysates assessed by Western blotting with antibodies against non-phosphorylated and phosphorylated (p-) AKT, ERK, and p38. Consistent with their known role in microglia differentiation, induction of p-AKT, p-ERK and p-p38 was observed in the cells stimulated with B1 Ab, but not with an isotype control (FIG. 5D). In addition to activation of transcription factors after binding to VIM, the B1Ab might be expected to induce phosphorylation of VIM itself. CD34⁺ cells were activated by the B1Ab and the degree of VIM phosphorylation was determined by Western blot using an antibody that detects phosphorylation of VIM at serine 38. The treated cells showed a marked increase in VIM phosphorylation starting at 5 minutes (FIG. 5E).

### Example 6 Microglia Induced by Antibody B have Anti-Inflammatory Phenotype

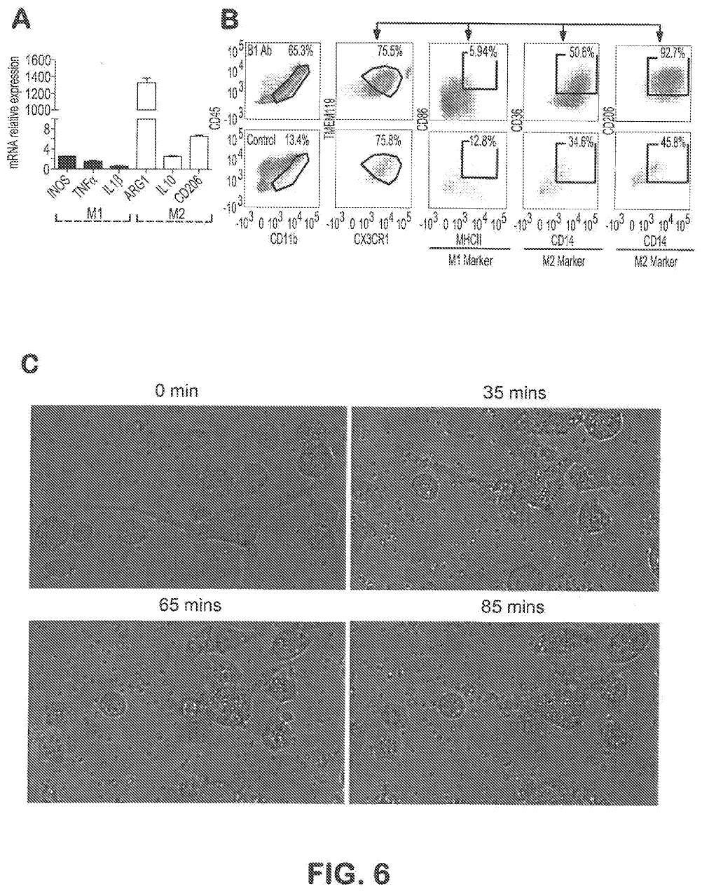

Polarization of the microglia is important because presumably one wants to induce those with anti-inflammatory properties. To determine the nature of the microglia induced by the antibody, total mouse bone marrow cells were incubated with the selected B antibody for two weeks in vitro, and specific M1/M2 marker gene mRNA and protein expression levels were analyzed by qRT-PCR and flow cytometry, respectively. Cells treated with B1 Ab up-regulated the M2 marker genes ARG1, IL10, and CD206, whereas expression of the M1 markers iNOS, TNFα, and IL1β remained low (FIG. 6A). Further, flow cytometric analysis revealed that the majority of the induced CD45^{low-int}CD11b⁺ cells stained positive for the microglial markers CX3CR1 and TMEM119 as well as the M2 markers CD14, CD36, and CD206, but negative for the M1 markers CD86 and MHCII (FIG. 6B). Together these data suggest that the B1 Ab induced the mouse bone marrow HSCs to differentiate into microglia with M2 polarization.

Since microglia are important phagocytic cells in the brain, a functional phagocytic assay was performed on the microglia produced from the in vitro differentiation of human CD34⁺ cells by the B1 Ab. The induced microglia were incubated with fluorescently labeled beads and monitored by RT-fluorescence microscopy for engulfment of beads over time. Marked phagocytosis of the beads by the induced microglia was seen and was most notable after 85 minutes of incubation (FIG. 6C). The cells were fixed after 85 minutes and the phagocytic cells were confirmed to be microglia by positive staining with mouse microglia-specific marker, IBA1. Active phagocytosis of the beads by microglia was observed and captured in a time-lapse movie.

We additionally performed Aβ peptide aggregation assays on the microglia induced from human CD34⁺ cells by the B1 Ab. This was intended to establish that the induced microglia-like cells are phagocytic in the therapeutic setting of Alzheimer's disease wherein the extension of this phagocytic function to the amyloid beta peptide (Aβ) is of central importance. In this study, we examined the ability of the microglia induced by the antibody to phagocytose Aβ (1-42). The results indicate that the cells were strongly phagocytic for a fluorescent derivative of Aβ (HIilyte Fluor 488).

### Example 7 the Induced Microglia-Like Cells Lower Aβ Deposition in the Brain

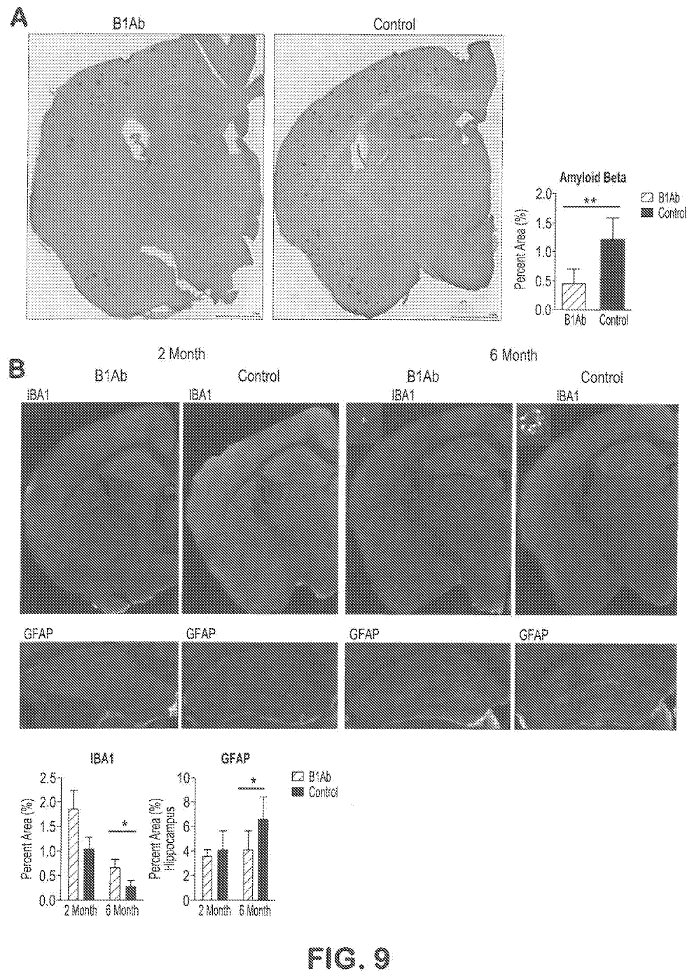

We next investigated whether antibody B1 could be used to lower Aβ plaques in the APP/PS1 Alzheimer's disease mouse model, whereby the mice develop Aβ plaques and Alzheimer's disease by 6 months of age. The gene for the B1 antibody was inserted into the genome of fresh bone marrow cells from wild-type mice. Then, these donor wild type bone marrow cells were adoptively transferred into irradiated APP/PS1 mice 8 weeks old mice. Brains were removed 10 days or five months after adoptive transfer. The brains were perfused and prepared for immunofluorescence histochemistry. At the 6 month time point, the brains of B1 Ab treated mice had a significant decrease in Aβ deposition as compared to control mice. Aβ deposition was 60% lower than control mice (FIG. 9A). In addition, at the 6 month time point animals treated with B1 Ab had more microglia and fewer astrocytes, which is consistent with reduced inflammation and less neurodegeneration (FIG. 9B).

### Example 8 Microglia-Like Cells Migrate to the Injured Brain in the Absence of Irradiation

In the studies above, brain irradiation was used as to increase the efficiency of the adoptive transfer. Thus, one could argue that irradiation was also necessary for migration of microglia to the brain and our studies would not, thus, be applicable to other types of brain injury such as Alzheimer's. Therefore, we carried out studies in aged APP/PS1 mice where bone marrow transfer was carried out without irradiation.

mCherry⁺ mouse bone marrow cells treated with B1 Ab were transplanted into non-irradiated 8 month old APP/PS1 mice and C57BL6 wild type mice. After 1 week, brain sections were stained with DAPI, IBA1, anti-mCherry and anti-Amyloid β antibodies. mCherry⁺ cells from BlAb treated bone marrow in these mice significantly migrated into the brains of aged APP/PS1 mice brains as compared to controls such as aged APP/PS1 mice that were not treated with B1 Ab and aged wild type mice. Importantly, the mCherry⁺ cells were found adjacent to plaques in the hippocampus that already contained abundant endogenous microglia. In summary, these studies suggest that the brain injury associated with Alzheimer's disease is a permissive condition and/or driving force that allows bone marrow cells to migrate to the brain where they are found at sites of injury.

### Example 9 Materials and Methods

Mouse strains and cell lines: The following mouse strains were used: C57BL/6J, B6 (Cg)-Tyr^{c-2J} Tg (UBC-mCherry) 1 Phbs/J, and 129S-Vim^{tm/Cba}/MesDmarkJ (The Jackson laboratory). The HEK293T cell line was maintained in DMEM medium containing 10% FCS, penicillin and streptomycin (Gibco-lnvitrogen). The Expi293F cell line was maintained in Expi293 Expression Media (Gibco-Invitrogen). Human CD34⁺ cells (All-Cells) and mouse bone marrow cells were cultured in StemSpan serum-free media with cytokine cocktail 100 (STEMCELL Technologies). Mice were housed and handled according to protocols approved by the Institutional Animal Care and Use Committee at The Scripps Research Institute. According to the Scripps Office for the Protection of Research Subjects Clinical Research Services, the study is not human subjects' research and does not require oversight by the Scripps Institutional Review Board.

Combinatorial antibody library and transduction: Single-chain Fv (ScFv) genes were obtained from a naïve human combinatorial antibody library (1×10¹¹ library diversity). ScFv genes were sub-cloned into a lentiviral vector. Lentivirus was produced in HEK293T cells by co-transfection of lentiviral vectors with the pCMVD8.91 and pVSVg viral packaging vectors at a ratio of 1:1:1. The mouse bone marrow cells were incubated with lentivirus for 3 days at 37° C.

Bone marrow transplantation: Bone marrow cells were transduced with the lentiviral antibody library at a multiplicity of infection of 2 and transplanted to lethally irradiated mice. The mice with transplanted bone marrow were maintained for 1-2 weeks. The brains were perfused, harvested, and kept frozen at −80° C. The antibody genes from the brain were amplified by PCR with primer pairs customized for our lentiviral vector, analyzed by electrophoresis, and recovered.

Purification of scFv-Fc proteins: The vector encoding the ScFv-Fc tag fusion protein was transfected into Expi293F cells for transient expression. Antibodies from the pooled supernatants were purified using HiTrap Protein G HP columns with an ÄKTAxpress purifier (GE). The buffer was exchanged to Dulbecco's PBS (pH 7.4) and stored at 4° C.

Immunoprecipitation and mass spectrometry: For immunoprecipitation, mouse bone marrow cells were prepared and solubilized in lysis buffer. The lysates were incubated with B1 Ab for 2 hours at 4° C., followed by incubation with 50 l of protein G-Sepharose beads (Pierce). The eluent was introduced into the linear trap quadrupole mass spectrometer from a nano-ion source with a 2-kV electrospray voltage. The analysis method consisted of a full MS scan with a range of 400-2,000 m/z followed by data-dependent MS/MS on the three most intense ions from the full MS scan. The raw data from the linear trap quadrupole were searched using the IPI human FASTA database with the MASCOT (http://www.matrixscience.com/) search engine.

Western blot: Cells were washed with PBS and then lysed in lysis buffer (50 mM Hlepes, pH 7.2, 150 mM NaCl, 50 mM NaF, 1 mM Na3VO₄, 10% glycerol, 1% Triton X-100). The lysates were then centrifuged at 12,000×g for 15 min at 4° C. The proteins were denatured in Laemmli sample buffer (5 min at 95° C.), separated by SDS/PAGE, and transferred to nitrocellulose membranes using the iBlot blotting system (Invitrogen). Membranes were blocked in phosphate buffered saline with Tween 20 (PBST) containing 5% BSA for 30 min before being incubated with antibodies for 3 h. VIM protein (Fitzgerald), C57BL/6J, and VIM-deficient mouse bone marrow lysates were used for identification. After washing the membranes several times with PBST, the blots were incubated with B1 Ab or horseradish peroxidase-conjugated anti-VIM or anti-β actin antibody for 1 h. The membranes were then washed with PBST and developed by ECL. Phosphorylation was performed with phospho-AKT, ERK and p38 (Cell Signaling Technology).

Flow cytometry and cell sorting: Cells were stained with anti-mouse CD11b, CD45, Ly6C, Ly6G, CD14, CD36, CD206, CD86, CD16/32, MHCII (BD Bioseciences), CX3CR1 (R&D system) and TMEM119 (kind gift from Dr. Barres, Stanford University). Stained cells were analyzed with a LSRII flow cytometer (Becton Dickinson).

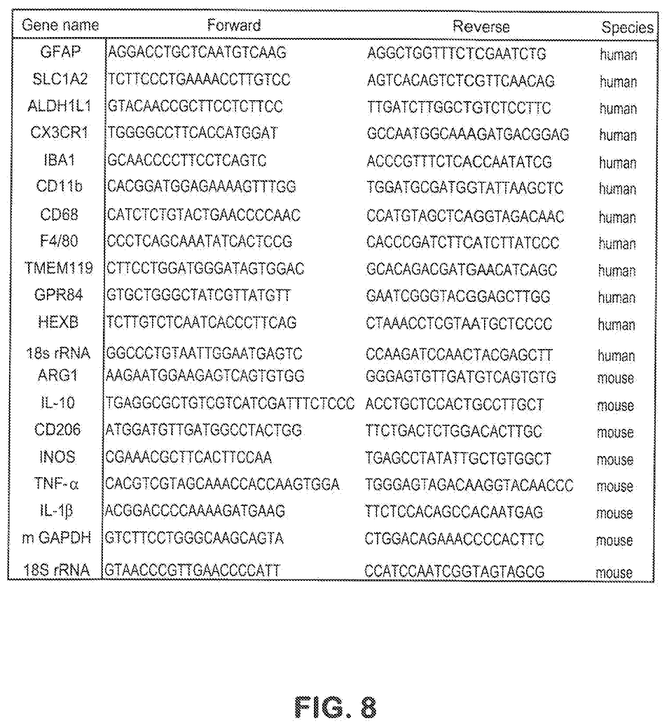

Real Time Quantitative (RT-q) PCR: RNA from cells cultured with B1 Ab was extracted (Qiagen) for cDNA synthesis (Bio-Rad Laboratories). PCR was performed in triplicate using 400 ng cDNA, the RT SYBR Green supermix, and a C1000 Thermal cycler (Bio-Rad Laboratories). Primer sets used were specific for human CX3CR1, IBA1, CD11b, CD68, F4/80, TMEM119, GPR84, HEXB, GFAP, SLCIA2, ALD1LA, Olig1, Olig2, and MOG, and for mouse ARG1, IL10, CD206, iNOS, TNFα, and IL1β. Primer sequences are shown in FIG. 8.

Immunohistochemistry and immunofluorescent confocal microscopy: Immunohistochemistry was performed on frozen brain sections. The brain section is a whole brain section and cut horizontally. Antibodies were diluted in 1×PBS containing 4% horse serum and 0.2% Triton-X100. Rat anti-mCherry (1:500, Invitrogen), goat anti-CX3CR1 (1:500, R&D system), rabbit anti-IBA1 (1:500, Wako), rat anti-CD11b (1:500, AbD serotec), or TMEM119 (kind gift from Dr. Barres, Stanford University) were used to detect markers for microglia. Sections were incubated overnight with primary antibodies. Sections were then incubated for 1 hour with secondary antibodies (goat anti-rabbit, goat anti-rat or donkey anti-goat, 1:250, Invitrogen). Immunofluorescent staining was performed on CD34⁺ cells, which were cultured on poly-L-lysine treated coverslips. Cells were fixed by 4% paraformaldehyde. Sections and coverslips were then mounted onto glass slides with anti-fade mounting medium with DAPI (ThermoFisher). Confocal microscopy was performed using a Zeiss LSM 710 laser scanning confocal microscope.

Bone marrow cells from luciferase-expressing transgenic mice (FVB-Tg (CAG-luc,-GFP) L2G85Chco/J) were transduced with the lentiviral B1 Ab and transplanted into lethally irradiated recipient mice (FVB/NJ). The mice were imaged 1 week post-transplantation. CycLuc (END Millipore) was injected (100 μl of 5 mM solution in PBS) i.v. into recipient mice prior to acquiring images using the IVIS Lumina® system (Perkin-Elmer). Images were acquired as 60 s exposure/image. Region of interest (ROI) were drawn around each brain, and the total number of counts within each ROI were recorded. Phagocytosis assay: The phagocytosis assay was conducted with DAPI labeled FluoSpheres Fluorescent Microspheres (Invitrogen). Human CD34⁺ cells were differentiated into microglia by the B1 Ab in a 6-well plate in vitro. Microbeads were sonicated and diluted (1:80) with RPMI medium (Invitrogen) without FBS. The diluted solution was then mixed with culture medium and incubated 2 hrs. To determine the phagocytic event, microglial engulfment was analyzed by an IN Cell Analyzer 6000 (GE) during incubation at 37° C.

The Aβ peptide aggregation assay was conducted with Beta—Amyloid (1-42) HiLyte™ Fluor 488—labeled (Anaspec). Human CD34⁺ cells were differentiated into microglia by the B1 Ab in a 6-well plate in vitro. Aβ peptide (20 μM) was mixed with culture medium and incubated for 12 hrs. Aβ peptide uptake experiment was analyzed by florescence microscopy (Zeiss).

Mouse brains were perfused, fixed in 4% paraformaldehyde for 24 h (4° C.), cryoprotected with 30% sucrose in PBS (4° C.), and frozen in dry ice. Serial coronal sections (50 μm thick) were collected from the genu of the corpus callosum to the caudal hippocampus. Sections (each separated by 300 μm) were stained with biotinylated HJ3.4 (Aβ 1-16) antibody (gift from Dr. Holtzman) to visualize Aβ-immunopositive plaques. Immunostained sections were imaged using a Leica scanner. Quantitative analysis of percent area covered by HJ3.4 was performed using the ImagePro program.

Total RNAs were isolated in replicates of three from untreated human CD34⁺ cells, human CD34⁺ cells treated with B1 Ab, and human CD34⁺ cells treated with M-CSF. Total RNA samples were prepared into RNAseq libraries using the NEBNext® Ultra™ Directional RNA Library Prep Kit for Illumina® following the manufacturer's recommended protocol. Briefly, for each sample 500 ng total RNA was polyA selected, converted to double stranded cDNA, followed by fragmentation and ligation of sequencing adapters. The library was then PCR amplified for 15 cycles using barcoded PCR primers, purified, and size selected using AMPure XP Beads before loading onto an Illumina NextSeq500 for 75 base single read sequencing. The expression levels of human transcripts were estimated using Salmon (BioRxiv). Statistical analyses were done with edgeR (Bioconductor), and the differentially expressed genes were identified as those with false-discovery rates <0.05, absolute fold change >2 and averaged CPM (counts per million) >1 in the samples. The heatmap was built using Cluster3 and JavaTreeView. Functional analysis of the differentially expressed genes was performed using Ingenuity Pathway Analysis software (Ingenuity Systems Inc.)

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A method for identifying a functional antibody that induce hematopoietic stem cell differentiation and migration to a specific organ or tissue, comprising (a) expressing in a population of stem cells a library of candidate antibodies or antigen-binding fragments thereof to produce a heterogeneous population of modified, antibody-expressing hematopoietic stem cells, (b) introducing the heterogeneous population of antibody-expressing hematopoietic stem cells into a non-human animal, and (c) detecting in a specific organ or tissue of the non-human animal the presence of a sequence encoding a candidate antibody; thereby identifying a functional antibody that induce stem cell differentiation and migration to said specific organ or tissue.

2. The method of claim 1, wherein the stem cells comprises bone marrow cells.

3. The method of claim 1, wherein the non-human animal is mouse.

4. The method of claim 1, wherein the non-human animal is lethally irradiated prior to introducing the antibody-expressing hematopoietic stem cells into the animal.

5. The method of claim 1, wherein the antibody-expressing hematopoietic stem cells are introduced into the animal via injection.

6. The method of claim 1, wherein the specific tissue is a tissue from brain, heart, liver or spleen.

7. The method of claim 1, wherein the library of candidate antibodies is a combinatorial library of scFv or scFv-Fc molecules.

8. The method of claim 7, wherein the combinatorial antibody library is expressed in the stem cells via a lentiviral vector or a retroviral vector.

9. The method of claim 1, further comprising determining amino acid sequences of heavy chain and light chain variable regions of the identified candidate antibody.

10. An antibody or an antigen-binding fragment that has the same binding specificity as that of a second antibody, wherein the second antibody comprises (1) heavy chain CDRs 1-3 and light chain CDRs 1-3 sequences that are respectively identical to GFN FNNYN (SEQ ID NO:4), ISSAADTV (SEQ ID NO:5), ARQLLY (SEQ ID NO:6), SGIN VGAYR (SEQ ID NO:7), YKSDSDK (SEQ ID NO:8), and AIWHSSAWV (SEQ ID NO:9), (2) heavy chain and light chain variable region sequences respectively shown in SEQ ID NOs:2 and 3, or (c) heavy chain and light chain variable region sequences respectively shown in SEQ ID NOs:11 and 12.

11. The antibody or antigen-binding fragment of claim 10, comprising heavy chain CDRs 1-3 sequences that are substantially identical, respectively, to (1) GFN FNNYN (SEQ ID NO:4), ISSAADTV (SEQ ID NO:5), and ARQLLY (SEQ ID NO:6) or (2) GFTFSSYA (SEQ ID NO:13), MSGSGGST (SEQ ID NO:14), and AKGVWFGELLPPFDY (SEQ ID NO: 15).

12. The antibody or antigen-binding fragment of claim 11, further comprising light chain CDRs 1-3 sequences that are substantially identical, respectively, to SGIN VGAYR (SEQ ID NO:7), YKSDSDK (SEQ ID NO:8), and AIWHSSAWV (SEQ ID NO:9).

13. The antibody or antigen-binding fragment of claim 10, comprising (1) a heavy chain CDR sequence selected from the group consisting of SEQ ID NOs:4-6 and 13-15; or (2) a heavy chain CDR sequence selected from the group consisting of SEQ ID NOs:4-6 and 13-15, except for conservative substitution at one or more residues in said heavy chain CDR.

14. The antibody or antigen-binding fragment of claim 13, further comprising (1) a light chain CDR sequence selected from the group consisting of SEQ ID NOs:7-9; or (2) a light chain CDR sequence selected from the group consisting of SEQ ID NOs:7-9, except for conservative substitution at one or more residues in said light chain CDR.

15. The antibody or antigen-binding fragment of claim 11, comprising heavy chain CDRs 1-3 sequences that are respectively identical to SEQ ID NOs:4-6 or SEQ ID NOs:13-15, except for conservative substitution at one or more residues in said heavy chain CDRs.

16. The antibody or antigen-binding fragment of claim 15, comprising heavy chain CDRs 1-3 and light chain CDRs 1-3 sequences respectively shown in SEQ ID NOs:4-6 and SEQ ID NOs:7-9, except for conservative substitution at one or more residues in said CDRs.

17. The antibody or antigen-binding fragment of claim 10, comprising a heavy chain variable region sequence that is at least 90% identical to SEQ ID NO:2 or 11.

18. The antibody or antigen-binding fragment of claim 10, comprising a light chain variable region sequence that is at least 90% identical to SEQ ID NO:3 or 12.

19. The antibody or antigen-binding fragment of claim 10, comprising a heavy chain variable region sequence and a light chain variable region sequence that are at least 90% identical, respectively, to (1) SEQ ID NOs:2 and 3 or (2) SEQ ID NOs:1 and 12.

20. The antibody or antigen-binding fragment of claim 10, comprising a heavy chain variable region sequence and a light chain variable region sequence, one or both of which are identical to a heavy chain variable region sequence and a light chain variable region sequence respectively shown in (1) SEQ ID NOs:2 and 3 or (2) SEQ ID NOs: 11 and 12.

21. The antibody or antigen-binding fragment of claim 20, comprising a heavy chain variable region sequence and a light chain variable region sequence respectively shown in (1) SEQ ID NOs:2 and 3; (2) SEQ ID NOs:2 and 3, except for conservative substitution at one or more residues therein; (3) SEQ ID NOs:11 and 12; or (4) SEQ ID NOs:11 and 12, except for conservative substitution at one or more residues therein.

22. The antibody or antigen-binding fragment of claim 10, which is a scFv fragment comprising heavy chain and light chain variable region sequences that are connected via a linker sequence.

23. The antibody or antigen-binding fragment of claim 22, wherein the linker sequence comprises GGGGGS (SEQ ID NO: 16) or GGGGSGGGGSGGGGS (SEQ ID NO:17).

24. The antibody or antigen-binding fragment of claim 22, comprising a sequence shown in (a) SEQ ID NO: i1, (b) SEQ ID NO: I1 except for conservative substitution at one or more residues therein, (c) SEQ ID NO: 10, or (d) SEQ ID NO:10 except for conservative substitution at one or more residues therein.

25. The antibody or antigen-binding fragment of claim 10, which is IgA1, IgA2, IgD, IgE, IgG1, IgG2, IgG3, IgG4, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depicting IgG, a diabody, or a bivalent antibody.

26. The antibody or antigen-binding fragment of claim 25, which is an IgG selected from the group consisting of IgG, IgG2, IgG3, IgG4, and synthetic IgG.

27. The antibody or antigen-binding fragment of claim 25, which is a Fab, a scFv, or a dsFv.

28. The antibody or antigen-binding fragment of claim 25, which is conjugated to an Fc domain or a label moiety.

29. A method for treating a brain disorder or injury in a subject, comprising administering to a subject afflicted with or at risk of developing the brain disorder or injury a pharmaceutical composition comprising a therapeutically effective amount of (1) the antibody of claim 10 or (2) a stem cell population that is first treated with the antibody of claim 10.

30. The method of claim 29, wherein the brain disorder is dementia.

31. The method of claim 29, wherein the stem cell population is isolated from the subject in need of treatment.

32. The method of claim 29, wherein the stem cell population comprises bone marrow cells.
